(19) United States
(12) Patent Application Publication
(10) Pub. No.: US 2026/0224706 A1
(43) Pub. Date: Aug. 6, 2026

(54) THERMOGELLING POLYMERS, FUNCTIONALIZED FORMS THEREOF, AND RELATED METHODS

(71) Applicant: AGENCY FOR SCIENCE, TECHNOLOGY AND RESEARCH, Singapore, (SG)

(72) Inventors: Xian Jun LOH, Singapore (SG); Zhi Rong Rubayn GOH, Singapore (SG); Yuan Chong Jason LIM, Singapore (SG)

(73) Assignee: AGENCY FOR SCIENCE, TECHNOLOGY AND RESEARCH, Singapore, (SG)

(21) Appl. No.: 19/155,070

(22) PCT Filed: Feb. 9, 2024
(86) PCT No.: PCT/SG2024/050074

§ 371 Date: Aug. 8, 2025

(30) Foreign Application Priority Data
Feb. 10, 2023 (SG) 10202300350S

(51) Int. Cl. A61K 47/34; C08G 18/48; C08G 18/67; C08G 18/73; C08G 18/83

(52) U.S. Cl. CPC A61K 47/34; C08G 18/4808; C08G 18/4825; C08G 18/4833; C08G 18/675; C08G 18/73; C08G 18/835

(57) ABSTRACT

There is provided a thermogelling polymer comprising one or more repeating units derived from a hydrophilic polymer, one or more repeating units derived from a thermoresponsive polymer, and one or more repeating units derived from a vinyl-containing diol monomer, wherein the one or more repeating units derived from a hydrophilic polymer, the one or more repeating units derived from a thermoresponsive polymer, and the one or more repeating units derived from a vinyl-containing diol monomer are chemically coupled together by at least one of urethane/carbamate, carbonate, ester linkages or combinations thereof. There are also provided a method of preparing said thermogelling polymer and a functionalized form of said thermogelling polymer.

### TECHNICAL FIELD

The present disclosure relates broadly to a thermogelling polymer, its functionalized forms, and related methods thereof.

### BACKGROUND

Thermogels or thermogelling copolymers are a group of thermo-responsive amphiphiles that undergo a temperature-dependent spontaneous sol-gel transition and self-assemble into a supramolecular gel when the temperature is raised, i.e., upon contact with the human body. This provides unique advantages like injectability, sprayability, and the ability to encapsulate temperature-sensitive therapeutics, drugs, proteins, or cells. Thus far, thermogels have been used for drug delivery and tissue engineering.

Presently, the ability to tailor the chemical composition of these synthetic gels has been limited to the incorporation of hydrolysable polyesters like poly(ε-caprolatone)-diol and poly(lactic-co-glycolic acid) (PLGA) to facilitate biodegradability. The high reactivity of isocyanates would preclude the incorporation of numerous chemical moieties along the polymer backbone in a facile manner, with control of functional group density, and without tedious protection and deprotection of functional groups. For example, existing approaches like chain-end modifications result in low functional group densities or lack the synthetic versatility that would allow for simple yet high-yielding functionalization of thermogels. Furthermore, these processes often require harsh conditions (e.g., concentrated corrosive acids).

In view of the above, there is a need to address or at least ameliorate the above-mentioned problems. In particular, there is a need to provide a method to conveniently allow post-synthetic chemical functionalisation of thermogelling polymers, which will imbue them with new properties.

### SUMMARY

In one aspect, there is provided a thermogelling polymer comprising one or more repeating units derived from a hydrophilic polymer, one or more repeating units derived from a thermoresponsive polymer, and one or more repeating units derived from a vinyl-containing diol monomer, wherein the one or more repeating units derived from a hydrophilic polymer, the one or more repeating units derived from a thermoresponsive polymer, and the one or more repeating units derived from a vinyl-containing diol monomer are chemically coupled together by at least one of urethane/carbamate, carbonate, ester linkages or combinations thereof,

In one embodiment, the one or more repeating units derived from a hydrophilic polymer is/are represented by general formula (1); the one or more repeating units derived from a thermoresponsive polymer is/are represented by general formula (2); the one or more repeating units derived from a vinyl-containing diol monomer is/are represented by general formula (3); and the at least one of urethane/carbamate, carbonate, ester linkages or combinations thereof is/are represented by general formula (5):

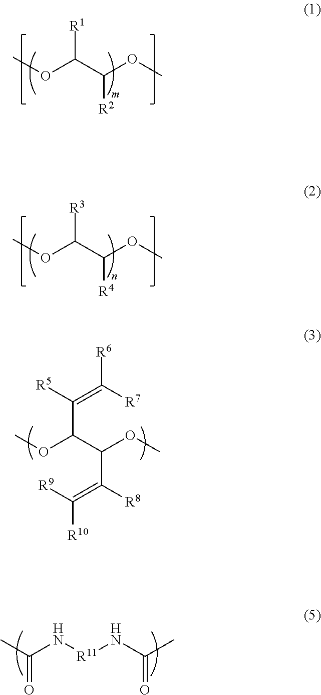

wherein
R¹ to R¹⁰ are each independently selected from H, alkyl, or optionally substituted alkyl;

$$m \geq 1 ;$$

$$n \geq 1 ;$$

R¹¹ is alkyl or optionally substituted alkyl.

In one embodiment, general formula (1) comprises poly(ethylene glycol) (PEG), general formula (2) comprises poly(propylene glycol) (PPG), general formula (3) comprises 1,5-hexadiene-3,4-diol (HDDO), and general formula (5) comprises hexamethylene diisocyanate (HMDI).

In one embodiment, general formula (1) is present in an amount of between 0 mass % to 90 mass % of the thermogelling polymer, general formula (2) is present in an amount of between 0 mass % to 90 mass % of the thermogelling polymer, general formula (3) is present in an amount of from 0.1 mass % to 40% of the thermogelling polymer, and general formula (5) is present in an amount of from 10 mass % to 60 mass % of the thermogelling polymer.

In one embodiment, the thermogelling polymer comprises 0.01 mmol/g to 7 mmol/g of vinyl groups (e.g., —CH═CH₂).

In one embodiment, the thermogelling polymer has a polydensity index (PDI) falling in a range of from 1 to 2.

In one embodiment, one or more vinyl groups of the thermogelling polymer have been further functionalized.

In one aspect, there is provided a functionalized thermogelling polymer comprising one or more repeating units derived from a hydrophilic polymer, one or more repeating units derived from a thermoresponsive polymer, and one or more repeating units derived from a functionalized vinyl-containing diol monomer,
wherein the one or more repeating units derived from a hydrophilic polymer, the one or more repeating units derived from a thermoresponsive polymer, and the one or more repeating units derived from a functionalized vinyl-containing diol monomer are chemically coupled together by at least one of urethane/carbamate, carbonate, ester linkages or combinations thereof.

In one embodiment, the one or more repeating units derived from a hydrophilic polymer is/are represented by general formula (1); the one or more repeating units derived from a thermoresponsive polymer is/are represented by general formula (2); the one or more repeating units derived from a functionalized vinyl-containing diol monomer is/are represented by general formula (4); and the at least one of urethane/carbamate, carbonate, ester linkages or combinations thereof is/are represented by general formula (5):

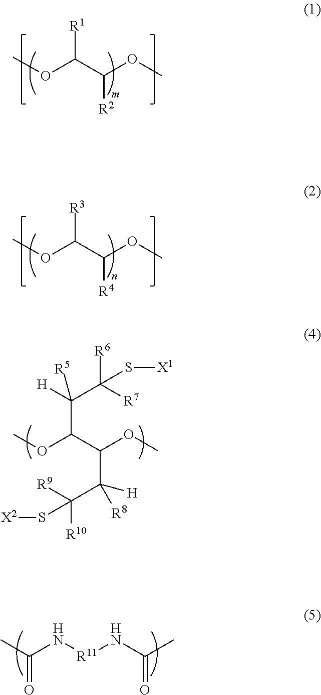

wherein
R¹ to R¹⁰, m and n are as defined above;
X¹—S and X²—S are each independently a thiol-containing functional group;
R¹¹ is alkyl or optionally substituted alkyl.

In one embodiment, general formula (1) comprises poly(ethylene glycol) (PEG), general formula (2) comprises poly(propylene glycol) (PPG), general formula (3) comprises 1,5-hexadiene-3,4-diol (HDDO), and general formula (5) comprises hexamethylene diisocyanate (HMDI).

In one embodiment, X¹ and X² are each independently a moiety comprising a carboxylic acid, an amino acid, a sulfonate, an alkylsulfonate, an aminehydrochloride, an alcohol, a diol, a benzene, an alkylbenzene, or derivatives thereof.

In one embodiment, X¹ and X² are each independently selected from the group consisting of the following structures or derivatives thereof:

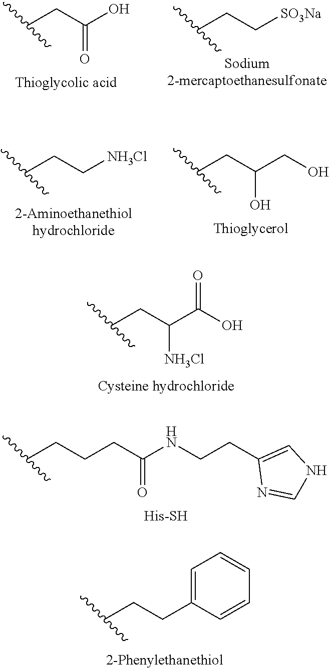

In one embodiment, the functionalized thermogelling polymer has one or more of the following properties: a pH value falling in a range of from 1 to 10; a gelation temperature falling in a range of from 4° C. to 60° C.; a crossover modulus falling in a range of from 5 Pa to 1000 Pa; a storage modulus (G′) in a range of from 4 Pa to 5000 Pa at about 37° C.; or a complex viscosity falling in a range of from 1 Pa·s to 500 Pa·s at about 37° C.

In one embodiment, the functionalized thermogelling polymer has a functional group density for X¹ and/or X² falling in a range of from 0.01 mmol/g to 7 mmol/g.

In one aspect, there is provided a method of preparing a thermogelling polymer, the method comprising:
coupling one or more hydrophilic polymers, one or more thermoresponsive polymers, and one or more vinyl-containing diol monomers in the presence of a coupling agent to obtain a thermogelling polymer,
wherein the coupling agent comprises a urethane/carbamate forming agent, carbonate forming agent, ester forming agent, or combinations thereof.

In one embodiment, the one or more hydrophilic polymers is/are represented by general formula (6); the one or more thermoresponsive polymers is/are represented by general formula (7); and the one or more vinyl-containing diol monomers is/are represented by general formula (8),

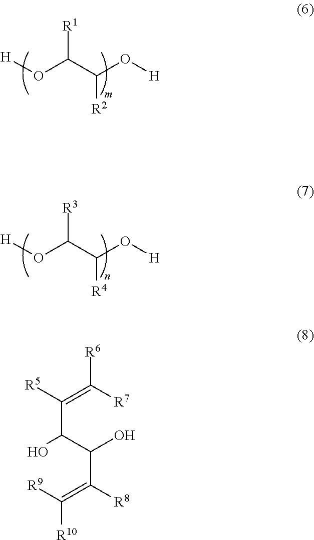

wherein R¹ to R¹⁰, m and n are as defined above.

In one embodiment, general formula (6) comprises poly(ethylene glycol) (PEG), general formula (7) comprises poly(propylene glycol) (PPG), and general formula (8) comprises 1,5-hexadiene-3,4-diol (HDDO).

In one embodiment, the one or more hydrophilic polymers represented by general formula (6), one or more thermoresponsive polymers represented by general formula (7), and one or more vinyl-containing diol monomers represented by general formula (8) are mixed in a mass ratio of 1-20:1-10:0.01-3.

In one embodiment, the coupling step is performed at an elevated temperature falling in a range of from 60° C. to 150° C.

In one embodiment, the coupling step is carried out for at least 2 hours.

In one embodiment, the coupling agent is a diisocyanate selected from the group consisting of hexamethylene diisocyanate (HMDI), tetramethylene diisocyanate, cyclohexane diisocyanate, tetramethylxylene diisocyanate, dodecylene diisocyanate, tolylene 2,4-diisocyanate, and tolylene 2,6-diisocyanate.

In one embodiment, the coupling agent is added in a ratio of 1-2:1-2 relative to one or more vinyl-containing diol monomers represented by general formula (8).

In one embodiment, the coupling step is carried out in the presence of an anhydrous solvent selected from the group consisting of toluene, benzene, and xylene.

In one embodiment, the coupling step is carried out in the presence of a metal-containing or non-metal containing catalyst that is capable of catalysing formation of urethane/carbamate, carbonate, ester linkages, or combinations thereof from alcohols and a suitable isocyanate precursor or derivatives thereof.

In one embodiment, the metal-containing catalyst may be a tin catalyst selected from the group consisting of alkyltin compounds, aryltin compounds, dialkyltin compounds such as dibutyltin dilaurate (DBTL), and a zinc-based catalyst such as zinc diethyldithiocarbamate.

In one embodiment, X¹—S and X²—S are each independently a thiol-containing functional group.

In one embodiment, the method further comprises functionalizing the thermogelling polymer with one or more thiols to obtain a functionalized thermogelling polymer with thiol-containing functional groups X¹—S and X²—S, where X¹ and X² each independently comprises a functional group.

In one embodiment, X¹ and X² are each independently a moiety comprising a carboxylic acid, an amino acid, a sulfonate, an alkylsulfonate, an aminehydrochloride, an alcohol, a diol, a benzene, an alkylbenzene, or derivatives thereof.

In one embodiment, X¹ and X² are each independently selected from the group consisting of the following structures or derivatives thereof:

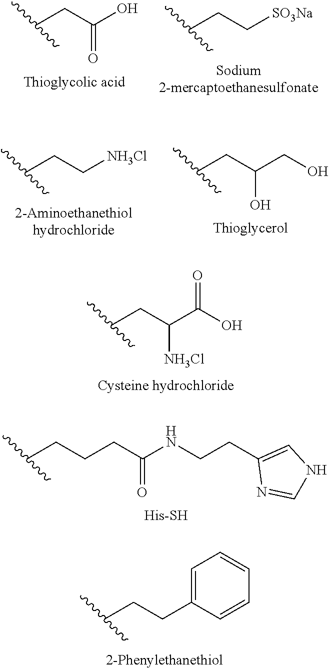

In one embodiment, functionalizing the thermogelling polymer is carried out in the presence of a radical initiator, photoinitiator, and/or ultraviolet (UV) light.

In one embodiment, functionalizing the thermogelling polymer is carried out in the presence of UV light for at least 30 minutes.

In one embodiment, functionalizing the thermogelling polymer is carried out in the presence of an anhydrous solvent, optionally wherein the anhydrous solvent is selected from the group of anhydrous tetrahydrofuran (THF) and anhydrous methanol.

In one embodiment, the one or more thiols is added in a molar ratio of 20-1:1-20, optionally 3:1, relative to the vinyl groups in the thermogelling polymer.

In one embodiment, the photoinitiator is added in a molar ratio of at least 3-1:1-3, optionally 2:1 relative to the vinyl groups in the functionalized thermogelling polymer.

In one embodiment, the photoinitiator comprises 2-hydroxy-4′-(2-hydroxyethoxy)-2-methylpropiophenone.

In one embodiment, functionalizing the thermogelling polymer with one or more thiols has a functionalization efficiency falling in a range of from 5% to 100%.

### Definitions

The terms “coupled” or “connected” as used in this description are intended to cover both directly connected or connected through one or more intermediate means, unless otherwise stated.

The term “alkyl” as a group or part of a group refers to a straight or branched aliphatic hydrocarbon group having 1 to 20 carbon atoms, 1 to 10 carbon atoms, 1 to 6 carbon atoms, or 1, 2, 3, 4, 5, 6, 7, 8, 9, 10, 11, 12, 13, 14, 15, 16, 17, 18, 19 or 20 carbon atoms. Examples of suitable straight and branched alkyl substituents include methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl and the like. The group may be a terminal group or a bridging group.

The term “optionally substituted,” when used to describe a chemical structure or moiety, refers to the chemical structure or moiety wherein one or more of its hydrogen atoms is optionally substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkyl sulfonyl, arylsulfonyl and arylalkylsulfonyl), sulfoxide, thiol (e.g., sulfhydryl, thioether) or urea (—NHCONH-alkyl-).

The term “derivative” as used herein refers to compounds that are derived from another compound and maintain the same general structure as the compound from which they are derived.

The term “functional group” as used herein refers to a group of atoms arranged in a way that determines the chemical properties of the group and the molecule to which it is attached. Examples of functional groups include but are not limited to carboxylic acid groups, amino acid groups, sulfonate groups, alkylsulfonate groups, aminehydrochloride groups, alcohol groups, diol groups, benzene groups, alkylbenzene groups, halogen atom containing groups, and the like.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

It will also be appreciated that where priority is claimed to an earlier application, the full contents of the earlier application is also taken to form part of the present disclosure and may serve as support for embodiments disclosed herein.

### DESCRIPTION OF EMBODIMENTS

Exemplary, non-limiting embodiments of a thermogelling polymer, its functionalized forms, and related methods of preparing these polymers are disclosed hereinafter.

In various embodiments, there is provided a thermogelling polymer comprising one or more repeating units derived from a hydrophilic polymer, one or more repeating units derived from a thermoresponsive/thermosensitive polymer, and one or more repeating units derived from a vinyl-containing diol monomer.

In various embodiments, the one or more repeating units derived from a hydrophilic polymer is/are represented by general formula (1), the one or more repeating units derived from a thermoresponsive polymer is/are represented by general formula (2), and the one or more repeating units derived from a vinyl-containing diol monomer is/are represented by general formula (3):

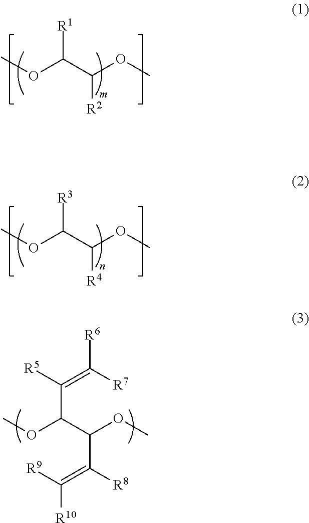

wherein
R¹ to R¹⁰ are each independently selected from H, alkyl, or optionally substituted alkyl;
m is from 1 to 400, from 5 to 395, from 10 to 390, from 15 to 385, from 20 to 80, from 25 to 375, from 30 to 370, from 35 to 365, from 40 to 360, from 45 to 355, from 50 to 350, from 60 to 340, from 70 to 330, from 80 to 320, from 90 to 310, from 100 to 300, from 110 to 290, from 120 to 280, from 130 to 270, from 140 to 260, from 150 to 250, from 160 to 240, from 170 to 230, from 180 to 220, from 190 to 210, or 200;
n is from 1 to 400, from 5 to 395, from 10 to 390, from 15 to 385, from 20 to 80, from 25 to 375, from 30 to 370, from 35 to 365, from 40 to 360, from 45 to 355, from 50 to 350, from 60 to 340, from 70 to 330, from 80 to 320, from 90 to 310, from 100 to 300, from 110 to 290, from 120 to 280, from 130 to 270, from 140 to 260, from 150 to 250, from 160 to 240, from 170 to 230, from 180 to 220, from 190 to 210, or 200.

In various embodiments, when m is approximately 340, the molecular weight of general formula (1) (e.g., PEG) is approximately 15 kDa.

In various embodiments, vinyl-containing diol monomers include but are not limited to 1,5-hexadiene-3,4-diol (HDDO), 3-(allyloxy) propane-1,2-diol, 2,3-dihydroxypropylmethacrylate, or the like. The vinyl-containing diol monomer may be a compound this is proprietary/self-synthesized or one that is commercially available.

In various embodiments, the thermogelling polymer is a multi-block polymer. The multi-block polymer may have at least one unit of the following structural sequence A-B-C, where A is a polymer block comprising at least one repeating unit represented by formula (1), B is a polymer block comprising at least one repeating unit represented by formula (2) and C is a polymer block comprising at least one repeating unit represented by formula (3). It may be appreciated that in some embodiments, the positions of A, B and C may be interchanged among themselves. In various embodiments, the multi-block polymer may comprise a plurality of repeating units represented by formula (1), a plurality of repeating units represented by formula (2) and/or a plurality of repeating units represented by formula (3). In various embodiments, the multi-block copolymer comprises more than 3 polymeric blocks. The blocks may be randomly distributed/arranged within the polymer.

In various embodiments, the one or more repeating units derived from a hydrophilic polymer is/are part of a hydrophilic polymer block and the one or more repeating units derived from a thermoresponsive/thermosensitive polymer is/are part of a thermoresponsive/thermosensitive polymer block.

In various embodiments, the polymer block comprising at least one repeating unit of formula (1), the polymer block comprising at least one repeating unit of formula (2) and the polymer block comprising at least one repeating unit of formula (3) are chemically coupled together by at least one of urethane/carbamate, carbonate, ester linkages or combinations thereof. For example, each of the polymer blocks are linked to their respective adjacent block by at least one of urethane/carbamate, carbonate, ester linkages or combinations thereof.

In various embodiments, the at least one of urethane/carbamate, carbonate, ester linkages or combinations thereof is/are represented by general formula (5):

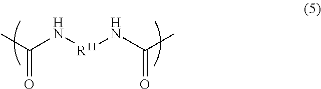

wherein R¹¹ is alkyl or optionally substituted alkyl.

In various embodiments, general formula (1) is different from general formula (2) in the thermogelling polymer. For example, general formula (1) may comprise PEG while general formula may comprise PPG. In various embodiments, general formula (5) comprises urethanes/carbamates. Thus, in various embodiments, general formula (1) comprises poly(ethylene glycol) (PEG), general formula (2) comprises poly(propylene glycol) (PPG), general formula (3) comprises 1,5-hexadiene-3,4-diol (HDDO), and general formula (5) comprises hexamethylene diisocyanate (HMDI).

In various embodiments, general formula (1) is present in an amount of falling in the range of between about 0 mass % and about 90 mass %, from about 5 mass % to about 85 mass % from about 10 mass % to about 80 mass %, from about 15 mass % to about 75 mass %, from about 20 mass % to about 70 mass %, from about 25 mass % to about 65 mass %, from about 30 mass % to about 60 mass %, from about 35 mass % to about 55 mass %, from about 40 mass % to about 50 mass %, of about 41 mass %, of about 42 mass %, of about 43 mass %, of about 44 mass %, of about 45 mass %, of about 46 mass %, of about 47 mass %, of about 48 mass %, of about 49 mass %, of about 50 mass %, of about 51 mass %, of about 52 mass %, of about 53 mass %, of about 54 mass %, of about 55 mass %, of about 56 mass %, of about 57 mass %, of about 58 mass %, of about 59 mass %, of about 60 mass %, of about 61 mass %, of about 62 mass %, of about 63 mass %, of about 64 mass %, or of about 65 mass % of the thermogelling polymer.

In various embodiments, general formula (2) is present in an amount of between about 0 mass % and about 90 mass %, from about 5 mass % to about 85 mass %, from about 10 mass % to about 80 mass %, from about 15 mass % to about 75 mass %, from about 20 mass % to about 70 mass %, from about 25 mass % to about 65 mass %, from about 30 mass % to about 60 mass %, from about 35 mass % to about 55 mass %, from about 40 mass % to about 50 mass %, from about 10 mass % to about 25 mass %, from about 11 mass % to about 24 mass %, from 12 mass % to about 23 mass %, from about 13 mass % to about 22 mass %, from about 14 mass % to about 21 mass %, from about 15 mass % to about 20 mass %, from about 16 mass % to about 19 mass %, from about 17 mass % to about 18 mass %, or about 17.5 mass % of the thermogelling polymer.

In various embodiments, general formula (3) is present in an amount of falling in the range of from about 0.1 mass % to about 40 mass %, from about 0.15 mass % to about 35 mass %, from about 0.2 mass % to about 30 mass %, from about 0.2 mass % to about 25 mass %, from about 0.2 mass % to about 20 mass %, from about 0.2 mass % to about 15 mass %, from about 0.2 mass % to about 10 mass %, from about 0.2 mass % to about 9.5 mass %, from about 0.2 mass % to about 9.0 mass %, from about 0.2 mass % to about 8.5 mass %, from about 0.2 mass % to about 8.0%, from about 0.5 mass % to about 7.5%, from about 1.0 mass % to about 7.0%, from about 1.5 mass % to about 6.5%, from about 2.0 mass % to about 6.0%, from about 2.5 mass % to about 5.5%, from about 3.0 mass % to about 5.0%, from about 3.5 mass % to about 4.5%, of about 4.0 mass %, of about 0.3 mass %, of about 0.31 mass %, of about 0.32 mass %, of about 0.33 mass %, of about 0.34 mass %, of about 0.35 mass %, of about 0.36 mass %, of about 0.37 mass %, of about 0.38 mass %, of about 0.39 mass %, of about 0.4 mass %, of about 8.0 mass %, of about 7.9 mass %, of about 7.8 mass %, of about 7.7 mass %, of about 7.6 mass %, of about 7.5 mass % of the thermogelling polymer. It will be appreciated that the mass % of general formula (3) present in the thermogelling polymer may vary with the molecular weight of the vinyl-containing diol monomer and the crosslinker used.

In various embodiments, general formula (5) is present in an amount of from about 10 mass % to about 60 mass %, from about 12 mass % to about 55 mass %, from about 15 mass % to about 50 mass %, from about 15 mass % to about 45 mass %, from about 15 mass % to about 40 mass %, from about 16 mass % to about 39 mass %, from about 17 mass % to about 38 mass %, from about 18 mass % to about 37 mass %, from about 19 mass % to about 36 mass %, from about 20 mass % to about 35 mass %, from about 21 mass % to about 34 mass %, from about 22 mass % to about 33 mass %, from about 23 mass % to about 32 mass %, from about 24 mass % to about 31 mass %, from about 25 mass % to about 30 mass %, from about 26 mass % to about 29 mass %, from about 27 mass % to about 28 mass %, or about 27.5 mass % of the thermogelling polymer. It will be appreciated that the mass % of general formula (5) present in the thermogelling polymer may vary with the molecular weight of the monomers and the crosslinkers used.

In various embodiments, the thermogelling polymer comprises from about 0.01 mmol/g to about 7.0 mmol/g, from about 0.02 mmol/g to about 6.0 mmol/g, from about 0.03 mmol/g to about 5.0 mmol/g, from about 0.04 mmol/g to about 4.0 mmol/g, from about 0.05 mmol/g to about 3.0 mmol/g, from about 0.05 mmol/g to about 2.0 mmol/g, from about 0.05 mmol/g to about 1.5 mmol/g, from about 0.10 mmol/g to about 1.45 mmol/g, from about 0.15 mmol/g to about 1.40 mmol/g, from about 0.20 mmol/g to about 1.35 mmol/g, from about 0.25 mmol/g to about 1.30 mmol/g, from about 0.30 mmol/g to about 1.25 mmol/g, from about 0.35 mmol/g to about 1.20 mmol/g, from about 0.40 mmol/g to about 1.15 mmol/g, from about 0.45 mmol/g to about 1.10 mmol/g, from about 0.50 mmol/g to about 1.05 mmol/g, from about 0.55 mmol/g to about 1.00 mmol/g, from about 0.60 mmol/g to about 0.95 mmol/g, from about 0.65 mmol/g to about 0.90 mmol/g, from about 0.70 mmol/g to about 0.85 mmol/g, from about 0.75 mmol/g to about 0.80 mmol/g, about 0.06 mmol/g, about 0.07 mmol/g, about 0.08 mmol/g, about 0.09 mmol/g, about 0.1 mmol/g, about 1.35 mmol/g, about 1.34 mmol/g, about 1.33 mmol/g, about 1.32 mmol/g, or about 1.31 mmol/g of vinyl groups (e.g., —CH═CH₂). Advantageously, in various embodiments, the vinyl groups allow for flexibility in the synthesis of a functionalized thermogelling polymer. For example, the vinyl groups may serve as a reactive handle/functionality to enable the incorporation of chemical moieties that impart additional functional properties or mechanical enhancements to the thermogelling polymer. In various embodiments, the weight percentage of vinyl groups incorporated into the thermogelling polymer can be tailored by controlling the initial amount of general formula (3) (e.g., HDDO) used in the reaction. It will be appreciated that the general formula (3) may comprise a modified vinyl-containing diol monomer such as a modified HDDO. As modifying the vinyl-containing diol monomer allows tunable hydrophobic-hydrophilic balance, thermogelling properties beyond the thermogelling polymer stated in the examples disclosed herein may be achieved. For example, when general formula (3) comprises HDDO, the HDDO may be tweaked to different polarities, such that the polymers subsequently obtained from these polymers may be capable of having higher vinyl content while retaining thermogelling properties than shown in the examples provided herein.

In various embodiments, the thermogelling polymer has a polydensity index (PDI) in a range of from about 1.0 to about 2, of from about 1.1 to about 1.9, of from about 1.2 to about 1.8, from about 1.3 to about 1.7, from about 1.4 to about 1.6, from about 1.45 to about 1.50, from about 1.31 to about 1.49, from about 1.32 to about 1.48, from about 1.33 to about 1.47, from about 1.34 to about 1.46, from about 1.35 to about 1.45, from about 1.36 to about 1.44, from about 1.37 to about 1.43, from about 1.38 to about 1.42, from about 1.39 to about 1.40, or about 1.395. In various embodiments, the upper limit of the PDI is accordance with the Carother's equation for polyaddition. Thus, due to nature of the type of polymerization that takes place, various embodiments of the presently disclosed polymer can result in a dispersity that is frequently large (e.g., up to a PDI of 2).

In various embodiments, the thermogelling polymer may further comprise other polymers and/or monomers that impart other desired properties to the thermogelling polymer. For example, the thermogelling polymer may comprise poly(caprolactone). Advantageously, in various embodiments, the poly(caprolactone) may provide biodegradability to the thermogelling polymer.

In various embodiments, there is provided a functionalized thermogelling polymer comprising one or more repeating units derived from a hydrophilic polymer, one or more repeating units derived from a thermoresponsive polymer, and one or more repeating units derived from a functionalized vinyl-containing diol monomer.

In various embodiments, the one or more repeating units derived from a hydrophilic polymer is/are represented by general formula (1), the one or more repeating units derived from a thermoresponsive polymer is/are represented by general formula (2), and the one or more repeating units derived from a functionalized vinyl-containing diol monomer is/are represented by general formula (4):

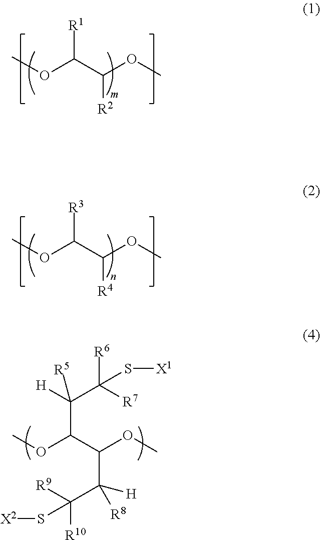

wherein
R¹ to R¹⁰, m and n are as defined above;
X¹—S and X²—S each independently comprises a thiol-containing functional group.

In various embodiments, the one or more repeating units derived from a hydrophilic polymer, the one or more repeating units derived from a thermoresponsive polymer, and the one or more repeating units derived from a functionalized vinyl-containing diol monomer are chemically coupled together by at least one of urethane/carbamate, carbonate, ester linkages or combinations thereof.

In various embodiments, the at least one of urethane/carbamate, carbonate, ester linkages or combinations thereof is/are represented by general formula (5):

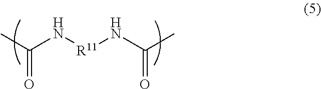

wherein R¹¹ is alkyl or optionally substituted alkyl.

In various embodiments, X¹ and X² may be each independently a moiety comprising one or more of a carboxylic acid, an amino acid, a sulfonate, an alkylsulfonate, an aminehydrochloride, an alcohol, a diol, a benzene, an alkybenzene, or the like or derivatives thereof. In various embodiments, X¹ and X² are each independently selected from the group consisting of the following structures or derivatives thereof:

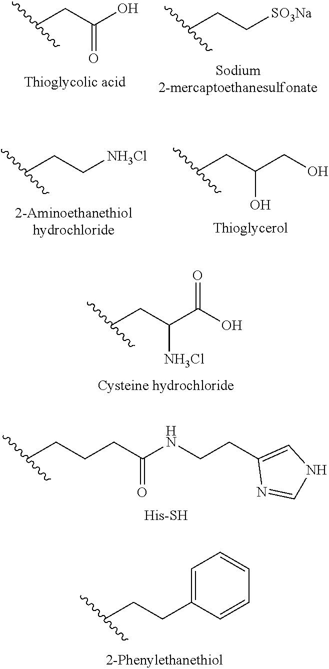

In various embodiments, the derivatives of the structures disclosed above include their ionized forms. It will be appreciated that other commercially available thiols such as mercaptoethanol, aromatic thiols (e.g., benzenethiol), or the like or derivatives thereof may also be used to obtain X¹—S and X²—S above.

It will be appreciated that the functionalized thermogelling polymer may comprise one or more properties and/or features of the thermogelling polymer previously described above or described herein forward.

In various embodiments, the functionalized thermogelling polymer may have a pH value in a range of from about 1 to about 10, from about 1.5 to about 9.5, from about 2.0 to about 8.0, from about 2.5 to about 7.5, from about 3.0 to about 7.0, from about 3.5 to about 6.5, from about 4.0 to about 6.0, from about 4.5 to about 5.5, or about 5.0.

In various embodiments, the functionalized thermogelling polymer may have a pH value that is substantially similar to physiological pH value ranging from about 7 to about 8, from about 7.1 to about 7.9, from about 7.2 to about 7.8, from about 7.3 to about 7.7, from about 7.4 to about 7.6, or about 7.5. In various embodiments, advantageously, the functionalized thermogelling polymer may be capable of demonstrating pH-responsive drug release. For example, functionalized thermogelling polymer functionalized with anionic carboxylate, anionic sulfonates, cationic amines, zwitterionic amino acid (e.g., cysteine) may exhibit pH-dependent sustained drug release and antifouling properties.

In various embodiments, advantageously, when the functionalized thermogelling polymer has a pH value beyond the physiological range, the functionalized thermogelling polymer may still be used in medical and/or non-medical applications. For example, the functionalized thermogelling polymer may be used in acidic environments such as stomach and/or cancer sites, or in the management of chronic wounds exhibiting alkaline pH levels.

In various embodiments, the functionalized thermogelling polymer has a critical gelation temperature/thermo-reversible sol-gel transition temperature/converts from a liquid/flowable state to a non-flowable/gel-like state at a temperature falling in a range of from about 4° C. to about 60° C., from about 5° C. to about 50° C., from about 6° C. to about 45° C., from about 7° C. to about 40° C., from about 7.5° C. to about 39° C., from about 8° C. to about 38° C., from about 8.5° C. to about 37° C., from about 9° C. to about 36° C., from about 9.5° C. to about 35° C., from about 10° C. to about 34° C., from about 15° C. to about 33° C., from about 20° C. to about 32° C., from about 21° C. to about 31° C., from about 22° C. to about 30° C., from about 23° C. to about 29° C., from about 24° C. to about 28° C., from about 25° C. to about 27° C., or of about 26° C., or at a temperature that is substantially similar to living human body temperature ranging from about 36° C. to about 37° C., or at about 37° C. For example, the functionalized thermogelling polymer may be in a liquid/flowable state at ambient room temperature (e.g., from about 20° C. to about 30° C.) and/or is in a non-flowable/gel-like state at living human body temperature (e.g., from about 36° C. to about 40° C.). Therefore, in some embodiments, the functionalized thermogelling polymer is in a flowable state at a temperature falling in the range of 20° C. to 30° C. and is in a non-flowable gel-like state at a temperature falling in the range of 30° C. to 37° C. Advantageously, in various embodiments, the functionalized thermogelling polymer is deliverable/injectable/sprayable or capable of encapsulating temperature-sensitive therapeutics, drugs, proteins, or cells as a liquid at 25° C. and gel at about 37° C. (with a viscosity range of from about 10 Pa·s to about 200 Pa·s). Even more advantageously, the functionalized thermogelling polymer may self-assemble into a supramolecular gel when the temperature is raised (i.e., upon contact with the human). In various embodiments, the thermogelling polymer remains inert after the functionalization. In various embodiments, the functionalization of the thermogelling polymer does not substantially affect its original gelation property. For example, the highly charged cationic and anionic polymers retain the capability to form gels at 37° C. For example, while the hydrophobic aromatic phenyl group increases the storage modulus of the thermogels at 37° C., they remain soluble at low temperatures. In various embodiments, when the functionalized thermogelling polymer has a gelation critical temperature/thermo-reversible sol-gel transition temperature/converts from a liquid/flowable state to a non-flowable/gel-like state at a temperature falling beyond the range of a temperature that is substantially similar to living human body temperature, the functionalized thermogelling polymer may be used in non-biological applications.

In various embodiments, the functionalized thermogelling polymer may have a crossover modulus falling in a range of from about 5 Pa to about 1000 Pa, from about 5 Pa to about 900 Pa, form about 5 Pa to about 800 Pa, from about 5 Pa to about 700 Pa, from about 5 Pa to about 600 Pa, from about 5 Pa to about 500 Pa, from about 5 Pa to about 400 Pa, from about 5 Pa to about 300 Pa, from about 5 Pa to about 200 Pa, from about 5 Pa to about 150 Pa, of from about 6 Pa to about 140 Pa, from about 7 Pa to about 130 Pa, from about 8 Pa to about 120 Pa, from about 9 Pa to about 115 Pa, from about 10 Pa to about 110 Pa, from about 20 Pa to about 100 Pa, from about 30 Pa to about 90 Pa, from about 40 Pa to about 80 Pa, from about 50 Pa to about 70 Pa, of about 11 Pa, of about 11.1 Pa, of about 11.2 Pa, of about 11.3 Pa, of about 11.4 Pa, of about 11.5 Pa, of about 11.6 Pa, of about 11.7 Pa, of about 11.8 Pa, of about 11.9 Pa, of about 12 Pa, of about 102 Pa, of about 101.9 Pa, of about 101.8 Pa, of about 101.7 Pa, of about 101.6 Pa, of about 101.5 Pa, of about 101.4 Pa, of about 101.3 Pa, of about 101.2 Pa, of about 101.1 Pa, or of about 101 Pa. It will be appreciated that the crossover modulus of the thermogelling polymer is dependent on its functional groups, catalyst, and polymer concentration.

In various embodiments, the functionalized thermogelling polymer may have a storage modulus (G′) falling in a range of from about 1 Pa to about 5000 Pa, from about 1 Pa to about 4000 Pa, from about 1 Pa to about 3000 Pa, from about 1 Pa to about 2000 Pa, from about 1 Pa to about 1000 Pa, from about 2 Pa to about 900 Pa, from about 3 Pa to about 800 Pa, of from about 4 Pa to about 700 Pa, from about 50 Pa to about 650 Pa, from about 100 Pa to about 600 Pa, from about 150 Pa to about 550 Pa, from about 200 Pa to about 500 Pa, from about 250 Pa to about 450 Pa, from about 300 Pa to about 400 Pa, of about 4.1 Pa, of about 4.2 Pa, of about 4.3 Pa, of about 4.4 Pa, of about 4.5 Pa, of about 4.6 Pa, of about 4.7 Pa, of about 4.8 Pa, of about 690 Pa, of about 689 Pa, or of about 688 Pa, or of about 350 Pa at 37° C. It will be appreciated that the storage modulus of the thermogelling polymer is dependent on its functional groups, catalyst, and polymer concentration. In various embodiments, thermogelling polymer functionalized with one or more hydrophobic aromatic phenyl groups may have an increased storage modulus at 37° C. while remaining soluble at low temperatures.

In various embodiments, the functionalized thermogelling polymer may have a complex viscosity falling in a range of from about 1 Pa·s to about 1000 Pa·s, from about 1 Pa·s to about 900 Pa·s, from about 1 Pa·s to about 800 Pa·s, from about 1 Pa·s to about 700 Pa·s, from about 1 Pa·s to about 600 Pa·s, from about 1 Pa·s to about 500 Pa·s, from about 1 Pa·s to about 400 Pa·s, from about 1 Pa·s to about 300 Pa·s, from about 1 Pa·s to about 200 Pa·s, from about 5 Pa·s to about 200 Pa·s, from about 10 Pa·s to about 200 Pa·s, from about 20 Pa·s to about 190 Pa·s, from about 20 Pa·s to 1 about 80 Pa·s, from about 30 Pa·s to about 170 Pa·s, from about 40 Pa·s to about 160 Pa·s, from about 50 Pa·s to about 150 Pa·s, from about 60 Pa·s to about 140 Pa·s, from about 70 Pa·s to about 130 Pa·s, from about 80 Pas to about 120 Pa·s, from about 90 Pa·s to about 110 Pa·s, of about 100 Pa·s, of about 1.5 Pa·s, of about 1.6 Pa·s, of about 1.7 Pa·s, of about 1.8 Pa·s, of about 119 Pa·s, of about 118 Pa·s, of about 117 Pa·s, of about 116 Pa·s, of about 115 Pa·s, or of about 114 Pa·s at 37° C. It will be appreciated that the complex viscosity of the thermogelling polymer is dependent on its functional groups, catalyst, and polymer concentration

In various embodiments, X¹ and/or X² have a functional group density falling in a range of from about 0.01 mmol/g to about 7 mmol/g, from about 0.01 mmol/g to about 6 mmol/g, from about 0.01 mmol/g to about 5 mmol/g, from about 0.01 mmol/g to about 4 mmol/g, from about 0.01 mmol/g to about 3 mmol/g, from about 0.01 mmol/g to about 2 mmol/g, from about 0.02 mmol/g to about 1.50 mmol/g, of from about 0.03 mmol/g to about 1.40 mmol/g, from about 0.05 mmol/g to about 1.35 mmol/g, from about 0.10 mmol/g to about 1.30 mmol/g, from about 0.15 mmol/g to about 1.25 mmol/g, from about 0.20 mmol/g to about 1.20 mmol/g, from about 0.25 mmol/g to about 1.15 mmol/g, from about 0.30 mmol/g to about 1.10 mmol/g, from about 0.35 mmol/g to about 1.05 mmol/g, from about 0.40 mmol/g to about 1.00 mmol/g, from about 0.45 mmol/g to about 0.95 mmol/g, from about 0.50 mmol/g to about 0.90 mmol/g, from about 0.55 mmol/g to about 0.85 mmol/g, from about 0.60 mmol/g to about 0.80 mmol/g, from about 0.65 mmol/g to about 0.75 mmol/g, of about 0.70 mmol/g, of about 0.031 mmol/g, of about 0.032 mmol/g, of about 0.033 mmol/g, of about 0.034 mmol/g, of about 0.035 mmol/g, of about 0.036 mmol/g, of about 0.037 mmol/g, of about 0.038 mmol/g, of about 0.039 mmol/g, of about 1.350 mmol/g, of about 1.349 mmol/g, of about 1.348 mmol/g, of about 1.347 mmol/g, or of about 1.348 mmol/g. Advantageously, the functional group density achieved through providing mid-chain functionalizble vinyl groups is higher than that of the existing strategy of introducing functional groups through the end-domains. It will be appreciated that the functional group density may be tuned by changing the number of vinyl groups introduced into the thermogelling polymer. In various embodiments, the functional group density may vary in accordance with functionalization of the vinyl-containing diol monomers (e.g., HDDO) with different hydrophobic/hydrophilic group. In various embodiments, the functional group density may vary in accordance with the ratio of the hydrophilic polymers to thermoresponsive/thermosensitive polymers in the functionalized thermogelling polymer.

In various embodiments, there is also provided a method of preparing a thermogelling polymer disclosed herein, the method comprising coupling one or more hydrophilic polymers, one or more thermoresponsive/thermosensitive polymers, and one or more vinyl-containing diol monomers in the presence of a coupling agent to obtain a thermogelling polymer.

In various embodiments, the one or more hydrophilic polymers is/are represented by general formula (6), the one or more thermoresponsive/thermosensitive polymers is/are represented by general formula (7), and the one or more vinyl-containing diol monomers is/are represented by general formula (8) together. The coupling step may be carried out in the presence of a coupling agent such that the one or more hydrophilic polymers represented by general formula (6), the one or more thermoresponsive/thermosensitive polymers represented by general formula (7), and the one or more vinyl-containing diol monomers represented by general formula (8) are chemically coupled/linked together by at least one of urethane/carbamate, carbonate, ester linkages or combinations thereof to form the thermogelling polymer:

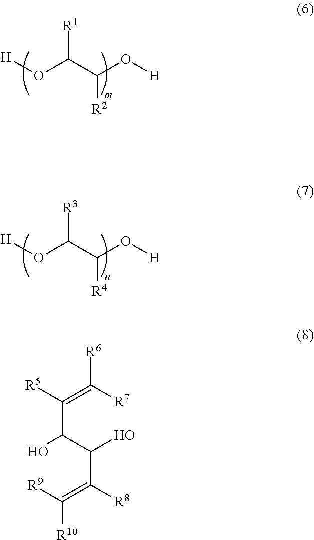

wherein R¹ to R¹⁰, m and n are as defined above.

In various embodiments, the at least one of urethane/carbamate, carbonate, ester linkages or combinations thereof is/are represented by general formula (5)

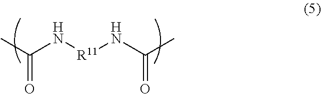

wherein R¹¹ is as defined above.

In various embodiments, general formula (6) is different from general formula (7) in the thermogelling polymer. For example, general formula (6) may comprise PEG while general formula (7) may comprise PPG. In various embodiments, general formula (5) comprises urethanes/carbamates. Thus, in various embodiments, general formula (6) comprises poly(ethylene glycol) (PEG), general formula (7) comprises poly(propylene glycol) (PPG), and general formula (8) comprises 1,5-hexadiene-3,4-diol (HDDO).

In various embodiments, the one or more hydrophilic polymers represented by general formula (6), the one or more thermoresponsive/thermosensitive polymers represented by general formula (7), and the one or more vinyl-containing diol monomers represented by general formula (8) are mixed in a mass ratio of about 1-20:1-10:0.01-3. For example, the mass ratio of the one or more hydrophilic polymers represented by general formula (6), the one or more thermoresponsive/thermosensitive polymers represented by general formula (7), and the one or more vinyl-containing diol monomers represented by general formula (8) may be about 3:1:0.01, about 3:1:0.10, about 3:1:0.20, about 3:1:0.30, about 3:1:0.40, about 3:1:0.50, about 3:1:0.51, about 3:1:0.52, about 3:1:0.53, about 3:1:0.54, about 3:1:0.55, about 3:1:0.56, about 3:1:0.57, about 3:1:0.58, about 3:1:1, about 3:1:2, about 3:1:3, about 3:1:4, or about 3:1:5. In various embodiments, the mass ratio of the one or more hydrophilic polymers represented by general formula (6) to the one or more thermoresponsive/thermosensitive polymers represented by general formula (7) is about 3:1, about 3:2, about 3:3, about 3:4, about 3:5, about 15:1, about 15:2, about 15:3, or about 15:4. It will be appreciated that functionalizing the vinyl-containing diol monomer (e.g., HDDO) with different mass ratios of hydrophobic and hydrophilic groups may affect the hydrophilic/hydrophobic balance and the thermogelling ability of the funcitonalized thermogelling polymer. For example, tailoring the ratio of PEG:PPG may allow a functionalized thermogelling polymer with a high HDDO content to be obtained.

In various embodiments, the coupling and/or mixing step is performed at an elevated temperature of from about 60° C. to about 150° C., from about 70° C. to about 150° C., from about 80° C. to about 150° C., from about 90° C. to about 150° C., from about 100° C. to about 150° C., from about 102° C. to about 148° C., 104° C. to about 146° C., from about 106° C. to about 144° C., from about 108° C. to about 142° C., from about 110° C. to about 140° C., from about 112° C. to about 138° C., from about 114° C. to about 136° C., from about 116° C. to about 134° C., from about 118° C. to about 132° C., from about 120° C. to about 130° C., from about 122° C. to about 128° C., from about 124° C. to about 126° C., or about 123° C. It will be appreciated that the temperature may vary according to the reaction time and catalysts used.

In various embodiments, the coupling and/or mixing step is carried out for up to about 36 hours, up to about 35 hours, up to about 30 hours, up to about 24 hours, up to about 20 hours, up to about 15 hours, up to about 10 hours, at least about 2 hours, at least about 3 hours, at least about 4 hours, or at least about 5 hours.

In various embodiments, the coupling and/or mixing step is performed in the absence of air and/or water/moisture and/or in the presence of a drying agent such as molecular sieves. Accordingly, in various embodiments, the absence of air and/or water/moisture may reduce or prevent occurrence of undesired side reactions. For example, the absence of air and/or water/moisture may reduce or prevent occurrence of premature termination of polymerization and allow thermogelling polymers of sufficient molecular weight to be obtained.

In various embodiments, the coupling agent comprises a urethane/carbamate forming agent, carbonate forming agent, ester forming agent, or combinations thereof. In various embodiments, the coupling agent comprises an isocyanate monomer that contains at least two (e.g. two or more) isocyanate functional groups. The coupling agent may be a diisocyanate selected from the group consisting of hexamethylene diisocyanate (HMDI), tetramethylene diisocyanate, cyclohexane diisocyanate, tetramethylxylene diisocyanate, dodecylene diisocyanate, tolylene 2,4-diisocyanate, tolylene 2,6-diisocyanate, or the like and combinations thereof. In various embodiments, the coupling agent may be a compound comprising a diisocyanate that is proprietary/self-synthesized or one that is commercially available. In various embodiments, the coupling agent may be devoid of an isocyanate such as precursors for non-isocyanate polyurethanes.

In various embodiments, the coupling agent is added in a ratio of about 1-2:about 1-2, about 1:1, about 1:1.01, about 1:1.02, about 1:1.03, about 1:1.04, about 1:1.05, about 1:1.06, about 1:1.07, about 1:1.08, about 1:1.09, about 1:1.10, about 1:1.2, about 1:1.3, about 1:1.4, about 1:1.5, about 1:1.6, about 1:1.7, about 1:1.8, about 1:1.9, about 1:2, about 1.10:1, about 1.2:1, about 1.3:1, about 1.4:1, about 1.5:1, about 1.6:1, about 1.7:1, about 1.8:1, about 1.9:1, about 2:1 relative to one or more monomers represented by general formulae (6), (7), and/or (8).

In various embodiments, the coupling and/or mixing step is carried out in the presence of a solvent. The solvent may comprise an anhydrous solvent selected from the group consisting of toluene, benzene, xylene, or the like and combinations thereof.

In various embodiments, the coupling and/or mixing step is carried out in the presence of a metal-containing or non-metal containing catalyst that is capable of catalysing formation of urethane/carbamate, carbonate, ester linkages, or combinations thereof e.g. from alcohols and a suitable isocyanate precursor or derivatives thereof. The metal-containing catalyst may comprise Lewis acid metals such as Bi³⁺, Fe³⁺, Zn²⁺, Sc³⁺, La³⁺, Ti⁴⁺, and Sn⁴⁺. In various embodiments, the metal-containing catalyst comprising Sn(IV) complexes is preferred. In various embodiments, the Lewis acid metals may be in any oxidation state. In various embodiments, the metal-containing catalyst may comprise a tin-based or zinc-based catalyst selected from the group consisting of alkyltin compounds, aryltin compounds, and dialkyltin diesters such as dibutyltin dilaurate (DBTL), dibutyltin diacetate, dibutyltin dioctanoate, dibutyltin distearate, zinc diethyldithiocarbamate or the like and combinations thereof. In various embodiments, the non-metal containing catalyst may be an organocatalyst.

In various embodiments, the method comprises functionalizing the thermogelling polymer with one or more thiols to obtain a functionalized thermogelling polymer with thiol-containing functional groups X¹—S and X²—S, where X¹ and X² each independently comprises a functional group.

In various embodiments, X¹ and X² may each independently be a moiety comprising one or more functional groups. The functional group may be a carboxylic acid, an amino acid, a sulfonate, an alkylsulfonate, an aminehydrochloride, an alcohol, a diol, a benzene, an alkylbenzene, or the like or derivatives thereof. In various embodiments, X¹ and X² may each be independently selected from the group consisting of of the following structures, or their derivatives thereof:

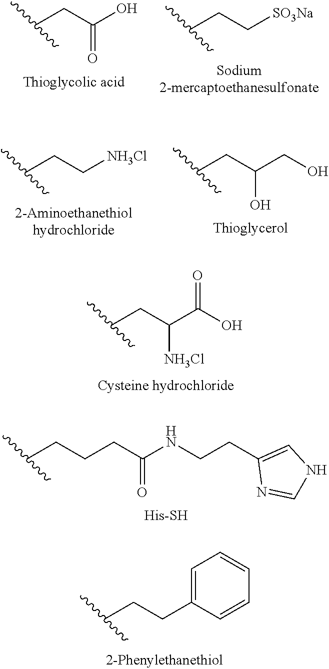

In various embodiments, derivatives of the above structures include their ionized forms, which may also be used. It will be appreciated that other commercially available thiols such as mercaptoethanol, aromatic thiols (e.g., benzenethiol), or the like or derivatives thereof may also be used to obtain the functional groups X¹—S and X²—S.

It will be appreciated that in various embodiments, the thermogelling polymer may be functionalized with multiple functional groups that are different or the same. For example, the vinyl groups of the thermogelling polymer may be functionalized with two or more of the structures for X¹ and X² described above.

In various embodiments, the wt % of the total thermogelling polymer that has been functionalized after the functionalizing step (e.g., click efficiency) falls in the range of from about 1 wt % to about 100 wt %, of at least about 5 wt %, of at least about 10 wt %, of at least about 15 wt %, of at least about 20 wt %, of at least about 25 wt %, of at least about 30 wt %, of at least about 35 wt %, of at least about 40 wt %, of about at least 45 wt %, of at least about 50 wt %, of at least about 55 wt %, of at least about 60 wt %, at least about 65 wt %, at least about 70 wt %, at least about 75 wt %, at least about 80 wt %, at least about 85 wt %, at least about 90 wt %, or at least about 95 wt %.

In various embodiments, the step of functionalizing the thermogelling polymer is carried out in the presence of a radical initiator, a photoinitiator and/or an irradiation source such as ultraviolet (UV) light or the like. In various embodiments, the functionalizing step is carried out for at least about 30 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, or at least about 12 hours, optionally in the presence of a radical initiator, a photoinitiator and/or an irradiation source such as ultraviolet (UV) light or the like.

In various embodiments, the step of functionalizing the thermogelling polymer with one or more thiols is carried out in the presence of an anhydrous solvent. In various embodiments, the anhydrous solvent may be selected from the group of anhydrous tetrahydrofuran (THF), anhydrous methanol, or the like and combinations thereof. It will be appreciated that the choice of the anhydrous solvent depends on the solubility of the one or more thiols used. For example, thiols such as sodium 2-mercaptoethanesulfonate, 2-aminoethanethiol hydrochloride, cysteine hydrochloride, and histamine-1-thiol (His-SH) may be dissolved in anhydrous methanol while thiols such as thioglycolic acid, thioglycerol, and 2-phenylethanethiol may be dissolved in anhydrous THF.

In various embodiments, the one or more thiols is added in a molar ratio of 20-1:1-20 relative to the vinyl groups in the thermogelling polymer. For example, the molar ratio of the one or more thiols added to the vinyl groups in the thermogelling polymer may be about 20:1, about 15:1, about 10:1, about 5:1, about 4:1, about 3:1, about 2:1, about 1:1, about 1:2, about 1:3, about 1:4, about 1:5, about 1:10, about 1:15, or about 1:20. Advantageously, in some embodiments, it was found that when the one or more thiols is added in 20 times excess, the functionalization step may reach completion, achieving a functionalization efficiency of 100%. In embodiments where the ratio of the thiols to the vinyl groups is low, a partial completion of thiol-ene click reaction may be leveraged to produce a thermogelling polymer comprising both vinyl groups and ionized form of X¹ and X² (e.g., cations).

In various embodiments, the radical initiator and/or photoinitiator is added in a molar ratio of at least 1-3:3-1 relative to the vinyl groups in the functionalized thermogelling polymer. For example, the molar ratio of the radical initiator and/or photoinitiator to the vinyl groups in the thermogelling polymer may be about 3:1, about 2:1, about 1:1, about 1:2, or about 1:3.

In various embodiments, the radical initiator comprises a photoinitiator, 2-hydroxy-4′-(2-hydroxyethoxy)-2-methylpropiophenone.

In various embodiments, the step of functionalizing the thermogelling polymer with one or more thiols has a functionalization efficiency (e.g., click efficiency) falling in a range of from about 5% to about 100%, from about 10% to about 95%, from about 15% to about 90%, from about 20% to about 85%, from about 25% to about 80%, from about 30% to about 75%, from about 35% to about 70%, from about 40% to about 65%, from about 45% to about 60%, from about 50% to about 55%, of at least about 20%, of at least about 21%, of at least about 22%, of at least about 23%, of at least about 24%, of at least about 25%, of at least about 26%, of at least about 27%, or of at least about 28%. It will be appreciated that in various embodiments, quantitative functionalization may be improved through greater excess use of thiols.

In various embodiments, the method further comprises the step of quenching the coupling reaction using a quenching agent (e.g., alcohols and its derivatives). In various embodiments, the quenching agent comprises ethanol. In various embodiments, the quenching step may be carried out for at least about 10 minutes, at least about 20 minutes, at least about 30 minutes, at least about 40 minutes, at least about 50 minutes, or at least about 1 hour. In various embodiments, the quenching step may be performed at an elevated temperature of no less than about 80° C., of no less than about 85° C., of no less than about 90° C., of no less than about 95° C., of from about 100° C. to about 150° C., from about 102° C. to about 148° C., 104° C. to about 146° C., from about 106° C. to about 144° C., from about 108° C. to about 142° C., from about 110° C. to about 140° C., from about 112° C. to about 138° C., from about 114° C. to about 136° C., from about 116° C. to about 134° C., from about 118° C. to about 132° C., from about 120° C. to about 130° C., from about 122° C. to about 128° C., from about 124° C. to about 126° C., or about 125° C.

In various embodiments, the method further comprises removing/purifying the synthesized thermogelling polymer and/or synthesized functionalized thermogelling polymer of/from contaminants via precipitation, filtering using a metal sieve, and/or drying overnight in the absence of air and/or water/moisture and/or in the presence of an inert gas such as nitrogen to remove unreacted reactants, solvents, and catalyst. In various embodiments, dialysis may be carried out at least about 3 times, at least about 4 times, or at least about 5 times with intervals of at least about 4 hours, at least about 5 hours, or at least about 6 hours before each change of dialysis buffer.

In various embodiments, the thermogelling polymer and/or functionalized thermogelling polymer disclosed herein may have a high water content of more than about 60%, about 65%, about 70%, about 75%, about 80%, about 85%, about 90%, about 95%, about 96%, about 97%, about 98%, or about 99% by weight. Accordingly, the thermogelling polymer may be a water-based polymer.

### BRIEF DESCRIPTION OF FIGURES

FIG. 1 is a schematic diagram for illustrating the application of hydrogels in drug delivery, tissue engineering, wound healing, and consumer cosmetics. Due to the high water content in hydrogels, hydrogels tend to be biocompatible while the polymeric scaffold could impart desirable properties such as mechanical integrity and bioactivity. This allows it to be used in drug delivery, wound healing, tissue engineering and in consumer cosmetics. However, a limitation of traditional hydrogels lies in their non-injectable and non-conformal nature. There is a need for the development of a versatile synthetic pathway to form multifunctional thermogels having functional handles that are chemically modifiable. To address this, thermo-responsive hydrogels (whereby the solution stays as a liquid at room temperature, but solidifies at 37° C. or upon body contact-thus making it injectable) have been developed in accordance with various embodiments disclosed herein. In various embodiments disclosed herein, the thermogelling polymers comprise hydrophilic PEG, hydrophobic PPG segments, functionalizable HDDO existing as a single unit or as segments, connected/coupled via HMDI linkers.

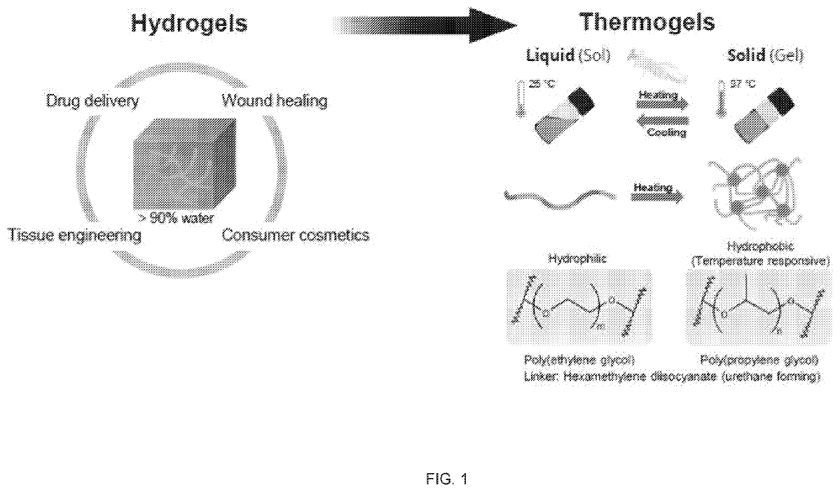

FIG. 2 shows ¹H NMR spectrum (in CDCl₃, 25° C.) of EP(7.7) V thermogelling polymer with 7.7 wt % of vinyl groups in accordance with an embodiment disclosed herein. Peaks [symbol] , [symbol] and ▪ indicate presence of PPG segment; peak ● indicates presence of PEG segment; peaks, ▴, ♦, and ★ indicate presence of HMDI moiety; and peaks a, b, and c indicate presence of reactive vinyl handles derived from integrating HDDO into polymer backbone. The spectrum shows the successful synthesis of EP(7.7)V thermogelling polymer, which comprises PEG, PPG, and HDDO linked via HMDI. Other peaks arising from HMDI moiety (i.e., OCONH and CH₂OCONH) and H₂O impurity can also be observed.

FIG. 3 shows ¹H NMR spectrum (in CDCl₃, 25° C.) of EP(7.7) V—COOH thermogelling polymer originally containing 7.7 wt % of vinyl groups that are subsequently functionalized with —COOH in accordance with an embodiment disclosed herein. Peaks [symbol] , [symbol] , and ≡ indicate presence of PPG segment; peak ● indicates presence of PEG segment; peaks ▴, ♦ and ★ indicate presence of HMDI moiety; peak a indicates presence of functional group —COOH. Absence of peak b in FIG. 4 indicates success of thiol-ene click reactions between the vinyl groups and thiolated substituents. The spectrum shows the successful functionalization of EP(7.7) V thermogelling polymer with —COOH in accordance with an example embodiment disclosed herein.

FIG. 4 shows ¹H NMR spectrum (in MeOD, 25° C.) of EP(7.7)V—SO₃Na thermogelling polymer originally containing 7.7 wt % of vinyl groups that are subsequently functionalized with —SO₃Na in accordance with an embodiment disclosed herein. Peaks [symbol] , [symbol] , and ▪ indicate presence of PPG segment; peak ● indicates presence of PEG segment; peaks, ▴, ♦ and ★ indicate presence of HMDI moiety; peaks a and b indicate presence of functional group —SO₃Na. Absence of peak b in FIG. 4 indicates success of thiol-ene click reactions between the vinyl groups and thiolated substituents. The spectrum shows the successful functionalization of EP(7.7) V thermogelling polymer with —SO₃Na in accordance with an example embodiment disclosed herein.

FIG. 5 shows ¹H NMR spectrum (in MeOD, 25° C.) of EP(7.7)V—NH₃Cl thermogelling polymer originally containing 7.7 wt % of vinyl groups that are subsequently functionalized with —NH₃Cl in accordance with an embodiment disclosed herein. Peaks [symbol] , [symbol] and ▪ indicate presence of PPG segment; peak ● indicates presence of PEG segment; peaks ▴, ♦ and ★ indicate presence of HMDI moiety; peak a indicates presence of functional group —NH₃Cl. Absence of peak b in FIG. 4 indicates success of thiol-ene click reactions between the vinyl groups and thiolated substituents. The spectrum shows the successful functionalization of EP(7.7) V thermogelling polymer with —NH₃Cl in accordance with an example embodiment disclosed herein.

FIG. 6 shows ¹H NMR spectrum (in MeOD, 25° C.) of EP(7.7)V-Cys thermogelling polymer originally containing 7.7 wt % of vinyl groups that are subsequently functionalized with -Cys in accordance with an embodiment disclosed herein. Peaks [symbol] , [symbol] , and ▪ indicate presence of PPG segment; peak ● indicates presence of PEG segment; peaks ▴, ♦ and ★ indicate presence of HMDI moiety; peaks a and b indicate presence of functional group -Cys. Absence of peak b in FIG. 4 indicates success of thiol-ene click reactions between the vinyl groups and thiolated substituents. The spectrum shows the successful functionalization of EP(7.7) V thermogelling polymer with -Cys in accordance with an example embodiment disclosed herein.

FIG. 7 shows ¹H NMR spectrum (in CDCl₃, 25° C.) of EP(7.7)V-Diol thermogelling polymer originally containing 7.7 wt % of vinyl groups that are subsequently functionalized with -Diol in accordance with an embodiment disclosed herein. Peaks [symbol] and [symbol] indicate presence of PPG segment; peak ● indicates presence of PEG segment; peaks ▴, ♦ and ★ indicate presence of HMDI moiety; peak a indicates presence of functional group -Diol. Absence of peak b in FIG. 4 indicates success of thiol-ene click reactions between the vinyl groups and thiolated substituents. The spectrum shows the successful functionalization of EP(7.7) V thermogelling polymer with -Diol in accordance with an example embodiment disclosed herein.

FIG. 8 shows ¹H NMR spectrum (in MeOD, 25° C.) of EP(7.7)V-Diol thermogelling polymer originally containing 7.7 wt % of vinyl groups that are subsequently functionalized with -His in accordance with an embodiment disclosed herein. Peaks [symbol] , [symbol] , and ▪ indicate presence of PPG segment; peak ● indicates presence of PEG segment; peaks ▴, ♦ and ★ indicate presence of HMDI moiety; peak a indicates presence of functional group -His. Absence of peak b in FIG. 4 indicates success of thiol-ene click reactions between the vinyl groups and thiolated substituents. The spectrum shows the successful functionalization of EP(7.7) V thermogelling polymer with -His in accordance with an example embodiment disclosed herein.

FIG. 9 shows ¹H NMR spectrum (in MeOD, 25° C.) of EP(7.7)V-Phenyl thermogelling polymer originally containing 7.7 wt % of vinyl groups that are subsequently functionalized with -Phenyl in accordance with an embodiment disclosed herein. Peaks [symbol] and [symbol] indicate presence of PPG segment; peak ● indicates presence of PEG segment; peaks ▴, ♦ and ★ indicate presence of HMDI moiety; peaks a to e indicate presence of functional group -Phenyl. Absence of peak b in FIG. 4 indicates success of thiol-ene click reactions between the vinyl groups and thiolated substituents. The spectrum shows the successful functionalization of EP(7.7) V thermogelling polymer with -Phenyl in accordance with an example embodiment disclosed herein.

FIG. 10 is an Ashby plot showing the changes in the storage moduli (G′) of an exemplary functionalized thermogelling polymers at 37° C. alongside their corresponding gelation temperatures. The Y axis represents the storage modulus, which is an indication of how stiff the gel is and the X axis being the temperature whereby the functionalized thermogel becomes a gel. EPV represents the PEG/PPG/vinyl-containing HDDO triblock thermogelling polymer before functionalization, with the bracketed number indicating the weight percentage of vinyl-containing monomers. EPV thermogelling polymer backbones comprising 0.4 wt %, 0.9 wt %, 4 wt %, and 7.7 wt % of HDDO subsequently functionalized with —COOH, —SO₃Na, NH₃Cl, Cys-HCl, phenyl, histamine, or diol were used. All the functionalized thermogelling polymers retained their thermogelling ability. Storage moduli and gelation temperatures were measured on a rheometer with a temperature sweep from 10° C. to 50° C. at 1 Hz.

### EXAMPLES

Example embodiments of the disclosure will be better understood and readily apparent to one of ordinary skill in the art from the following examples, tables and if applicable, in conjunction with the figures. It should be appreciated that other modifications related to structural, and/or chemical changes may be made without deviating from the scope of the invention. Example embodiments are not necessarily mutually exclusive as some may be combined with one or more embodiments to form new example embodiments. The example embodiments should not be construed as limiting the scope of the disclosure.

In examples below, an unprecedented versatile method for the synthesis of a library of multifunctional thermo-responsive random block-polyurethane copolymers is described. The polymers used in the examples are composed of urethane-linked segments of Poly(Ethylene glycol), Poly(Propylene glycol) and, Vinyl-containing 1,5-hexadiene-3,4-diol (EPV). By the incorporation of vinyl groups, the functional diversity of these thermo-responsive polymers is sought to be considerably expanded. These vinyl groups serve as a reactive handle to enable the incorporation of chemical moieties that impart additional functional properties or mechanical enhancements. By leveraging the versatility of thiol-ene click chemistry, the present inventors have now been able to functionalize these thermogels in one step with a broad range of commercially-available thiol-containing functional groups (TcFG). These functional groups inherently change the chemical and mechanical properties of the hydrogel, while allowing it to retain its thermogelling properties-allowing unprecedented ease in bottom-up engineering of a large family of diverse thermogels, influencing its functional properties and expanding its potential applications.

The following examples demonstrate (1) the synthesis of vinyl-containing thermogels, (2) the ease of post-polymerization functionalization using thiol-ene click chemistry, and (3) the thermogelling capabilities of the functionalized polyurethane copolymers.

### Example 1: Synthesis and Characterization of Thermogelling Polymer

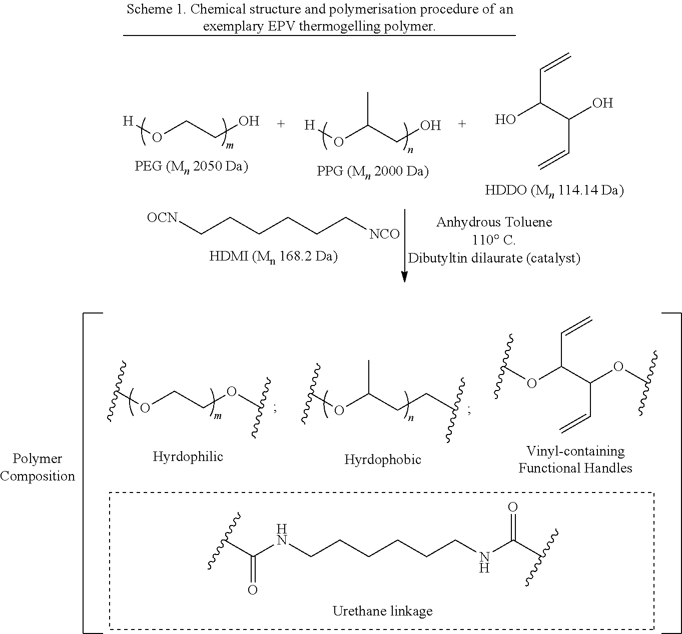

The general steps for preparing a thermogelling polymer in accordance with various embodiments disclosed herein include: coupling one or more polymers represented by general formula (6) (in the example below, PEG was used); one or more polymers represented by general formula (7) (in the example below, PPG was used); and one or more monomers represented by general formula (8) (in the example below, HDDO was used) in the presence of a coupling agent (in the example below, HMDI was used), a suitable catalyst (in the example below, DBTL was used), and a suitable solvent (in the example below, anhydrous toluene was used), as shown in Scheme 1 to obtain a thermogelling polymer.

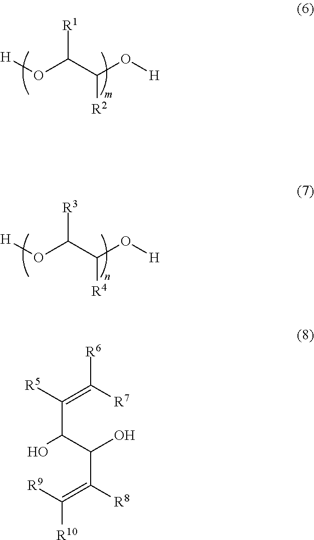

An example of preparing a thermogelling polymer designed in accordance with various embodiments disclosed herein is described in detail as follows.

### Materials

Anhydrous acetonitrile, anhydrous tetrahydrofuran, anhydrous methanol, 4 Å Molecular sieves (8-12 mesh), polyethylene glycol (Mn ~2050), polypropylene glycol (Mn ~2000), hexamethylene diisocyanate, dibutyltin dilaurate, and histamine were purchased from Sigma-Aldrich. 1,5-hexadiene-3,4-diol, thioglycolic acid, sodium mercaptoethane sulfonate, aminoethane hydrochloride, phenylethanethiol, thioglycerol and g-thiobutyrolactone from Tokyo Chemical Industry. Anhydrous toluene was purchased from Tedia and Diethyl ether from Fisher.

### Methods

Firstly, 4 Å molecular sieves were washed thrice in excess acetone, air-dried, and further activated under vacuum at 130° C. for over 15 hours. Subsequently, a 1000 mL 2-neck round bottom flask was filled with roughly 90 g of molecular sieves and approximately 500 mL of commercially available anhydrous toluene, and the mixture was left to dry for more than 24 hours. In parallel, macromonomers PEG (Mn=2050 Da) and PPG (Mn=2000 Da) were weighed into a 2-neck round bottom flask with a stir bar and the side arm was sealed with a rubber septum. Then, approximately 20 mL of anhydrous toluene was introduced to the flask, and azeotropic distillation was conducted at 60° C. under a reduced pressure of less than 55 mPa using a rotary evaporator for 15 minutes. This process was repeated twice. The macromonomers were then subjected to further drying under high vacuum at 110° C. for 1 hour. Next, in the same round bottom flask, 100 mL of anhydrous toluene and catalytic amount of dibutyltin dilaurate were added. Thereafter, HDDO (Mn=114.14 Da) was added to the solution with the mass ratio of the reactants being 3 PEG: 1 PPG: 0-0.58 HDDO respectively, followed by the introduction of HMDI (Mn=168.2 Da) in a 1 diol: 1.01 HMDI ratio to initiate the polyaddition polymerization. The polymerization was allowed to proceed for 2 hours at 110° C. To terminate the polymerization, 5 mL of ethanol was added and kept at 110° C. for 30 minutes. The resulting EPV thermogelling polymer was precipitated in diethyl ether at a ratio of 1:15, further filtered using a metal sieve, and dried overnight under nitrogen.

It will be appreciated that alternatively, the anhydrous toluene, DBTL, and HDDO may be added simultaneously. It will be understood that the anhydrous toluene, DBTL, and HDDO may be added in any other sequence and the order of addition of these reagents may not be substantially affect the outcome as long as the HMDI has not been added to initiate the polymerization.

The compositions and amount of vinyl groups per gram of the thermogelling polymer (mol/g) were determined using nuclear magnetic resonance (NMR) spectroscopy. The compositions were subsequently used to determine the stoichiometric ratios for the functionalization of vinyl groups. Table 1 shows the reaction and NMR compositions of each EPV thermogelling polymer with different weight percentage of vinyl groups. Table 2 shows the molecular weight and composition characterization of EPVs described in Table 1.

TABLE 1
Reaction compositions of EPV syntheses and resulting polymer compositions
verified by NMR. EPV samples are named with the weight percentage
of HDDO in parentheses. EP(0)V was used as a control.

| S/N | Composition (wt %) | PEG | PPG | HDDO | HMDI | Sample Name |
|---|---|---|---|---|---|---|
| 1 | Reaction composition | 69.23 | 23.07 | 0.00 | 7.70 | EP(0)V |
| | Polymer composition | 75.63 | 18.74 | 0.00 | 5.62 | |
| | by NMR | | | | | |
| 2 | Reaction composition | 68.36 | 22.79 | 0.5 | 8.35 | EP(0.4)V |
| | Polymer composition | 61.56 | 18.74 | 0.38 | 19.32 | |
| | by NMR | | | | | |
| 3 | Reaction composition | 67.50 | 22.50 | 1.00 | 9.00 | EP(0.9)V |
| | Polymer composition | 60.41 | 20.28 | 0.92 | 18.49 | |
| | by NMR | | | | | |
| 4 | Reaction composition | 60.54 | 20.18 | 5.04 | 14.24 | EP(4)V |
| | Polymer composition | 52.94 | 16.95 | 4.00 | 26.11 | |
| | by NMR | | | | | |
| 5 | Reaction composition | 51.93 | 17.31 | 10.04 | 20.72 | EP(7.7)V |
| | Polymer composition | 42.32 | 13.55 | 7.68 | 35.45 | |
| | by NMR | | | | | |

TABLE 2
Molecular weight and composition characterization of EPVs described in Table 1.

| | Date of | | | | | | | | | |
|---|---|---|---|---|---|---|---|---|---|---|
| | synthesis | Reaction | | M_{w} | | Composition (NMR, mass %) | | | | mmol/gram - |
| S/N | (DDMMYY) | duration (h) | Mₐ (kDa) | (kDa) | PDI | PEG | PPG | Hexadiene diol | HMDI | CH₂═CH₂ |
| 1 | 25 Jan. 2022 | 2 | 86.84 | 117.09 | 1.35 | 75.63% | 18.74% | 0% | 5.62% | 0 |
| 2 | 18 Jan. 2022 | 2 | 66.41 | 90.50 | 1.36 | 61.56% | 18.74% | 0.38% | 19.32% | 0.067 |
| 3 | 18 Jan. 2022 | 2 | 64.29 | 87.03 | 1.35 | 60.41% | 20.28% | 0.92% | 18.49% | 0.162 |
| 4 | 20 Jan. 2022 | 2 | 69.68 | 95.31 | 1.37 | 52.94% | 16.95% | 4.00% | 26.11% | 0.702 |
| 5 | 20 Jan. 2022 | 2 | 55.48 | 80.19 | 1.45 | 42.32% | 13.55% | 7.68% | 36.45% | 1.346 |

This versatile platform for the synthesis of multifunctional thermogels incorporates vinyl-containing diols, with amphiphilic dihydroxy-terminated macromonomers via addition polymerization in the presence of diisocyanates. The enabling strategy, therefore, results in a polymer that could be easily functionalized using thiol-ene click chemistry.

### Example 2: Synthesis and Characterization of Functionalized Thermogelling Polymer

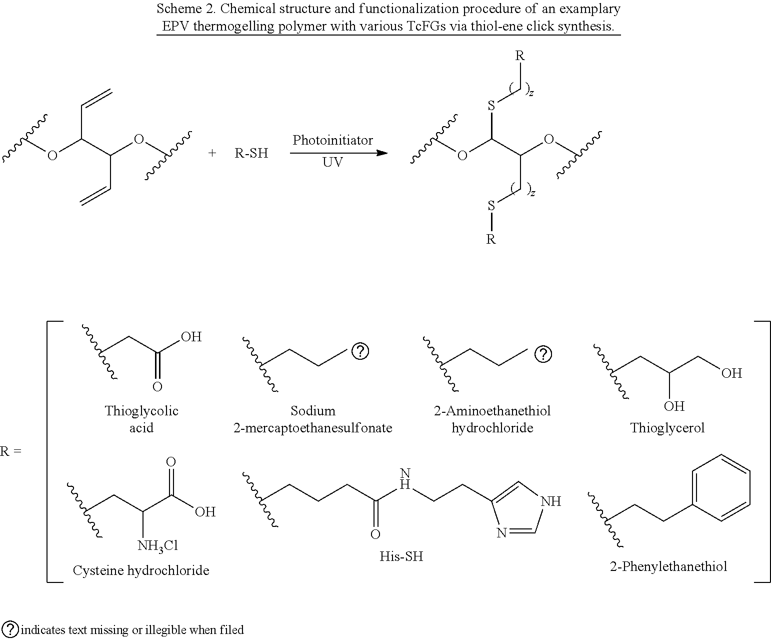

The general steps for functionalizing a thermogelling polymer in accordance with various embodiments disclosed herein include: functionalizing the thermogelling polymer (in the example below, EP(0.9) V was used) with one or more thiols in the presence of a photoinitiator (in the example below, 2-hydroxy-4′-(2-hydroxyethoxy)-2-methylpropiophenone was used) and/or ultraviolet (UV) light (in the example below, UV light with a wavelength of 365 nm was used) as shown in Scheme 2 to obtain a functionalized thermogelling polymer with functional moieties (in the example below, thioglycolic acid (anionic), sodium 2-mercaptoethanesulfonate (anionic), an acid, and an acid salt with pKa's of about 5 and about-7, 2-aminoethanethiol hydrochloride (cationic), cysteine hydrochloride (zwitterionic), 2-phenylethanethiol (aromatic), His-SH (imidazole, metal-binding), and thioglycerol (dihydroxy, hydrogen-bonding) were used) X¹—S and X²—S, where X¹ and X² are each independently a moiety comprising a carboxylic acid, an amino acid, a sulfonate, an alkylsulfonate, an aminehydrochloride, an alcohol, a diol, a benzene, an alkylbenzene or derivatives thereof and X¹ and X² correspond to R in Scheme 2.

An example of functionalizing a thermogelling polymer designed in accordance with various embodiments disclosed herein is described in detail as follows.

Firstly, 1 g of EP(0.9) V was introduced into a 20 mL vial with a septum and a stir bar. Following this, a photoinitiator, 2-hydroxy-4′-(2-hydroxyethoxy)-2-methylpropiophenone, was added in a 2:1 vinyl to photoinitiator molar ratio, while TcFG was added in a 1:3 vinyl to TcFG molar ratio. Depending on the solubility of TcFG, 10 mL of anhydrous methanol or THF was added to the vial to obtain a solution with a concentration of 100 mg/mL. For example, methanol-soluble TcFGs include sodium 2-mercaptoethanesulfonate, 2-aminoethanethiol hydrochloride, cysteine hydrochloride, and His-SH, while THF-soluble TcFGs include thioglycolic acid, thioglycerol, and 2-phenylethanethiol. The reactants were dissolved under continuous stirring on a magnetic stirrer. Next, the solution was sparged with argon for 10 minutes, and then exposed to an 8 W UV lamp (\ =365 nm, UVP, UVLS-28) for 3 hours with stirring. Subsequently, the resulting functionalized polymer was precipitated in diethyl ether in a 1:10 ratio, filtered using a metal sieve, and dried overnight under nitrogen. The resulting polymer was redissolved in MilliQ water (or methanol for His-SH) and dialyzed against MilliQ water (or methanol for His-SH) for a total of three times, with a minimum of 4 hours before each change of the dialysis buffer. Finally, the dialyzed sample was freeze-dried to obtain a functionalized thermogelling polymer.

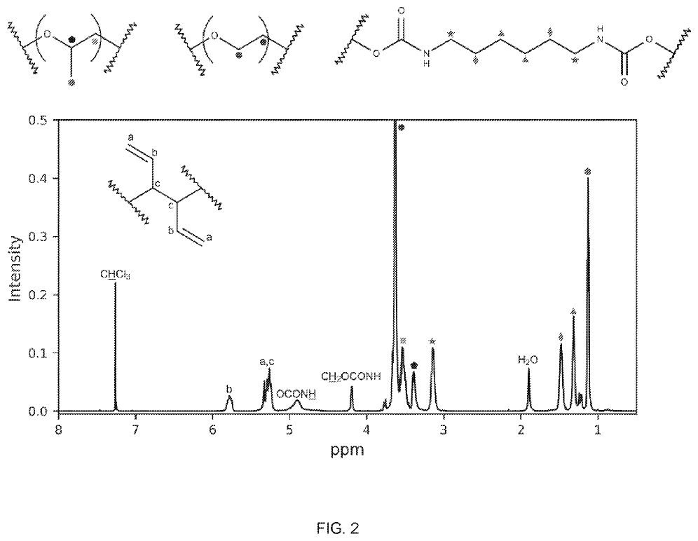

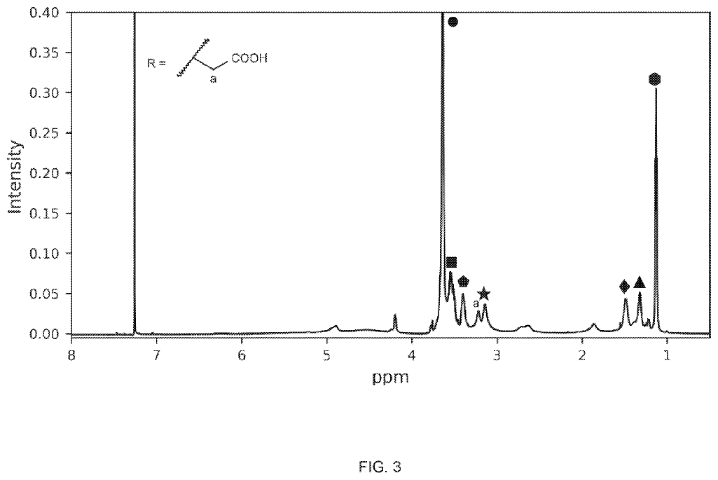

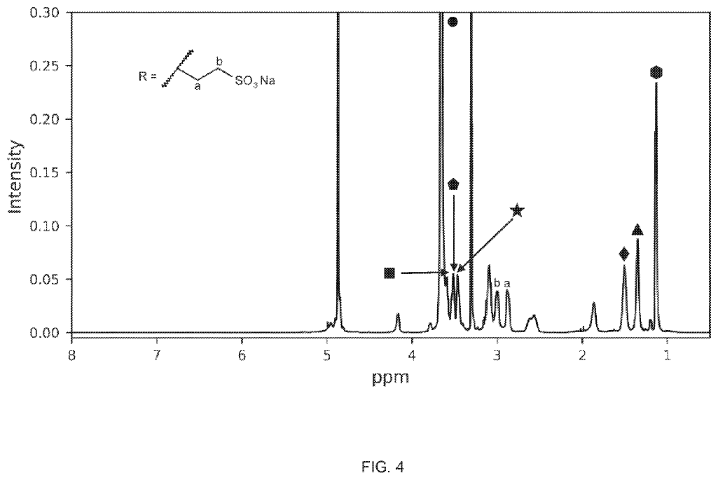

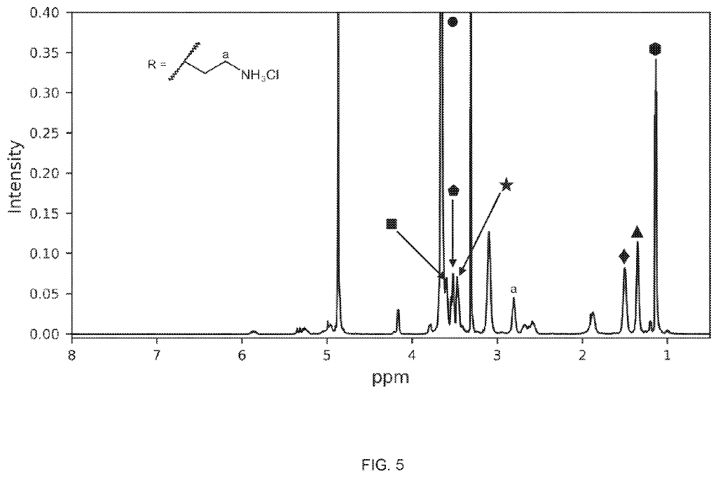

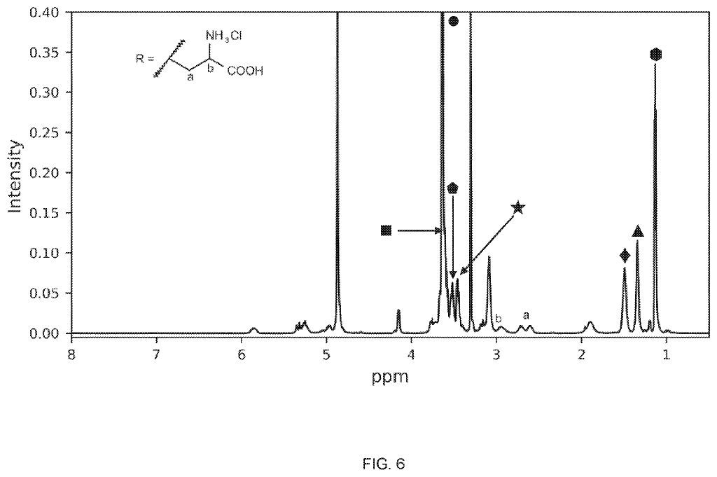

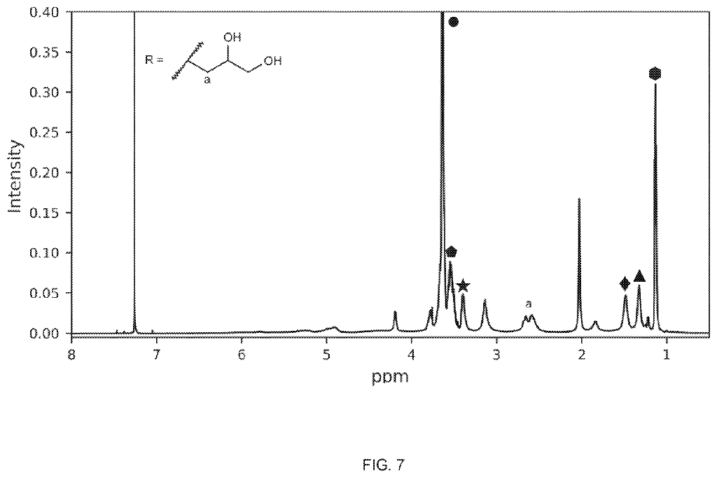

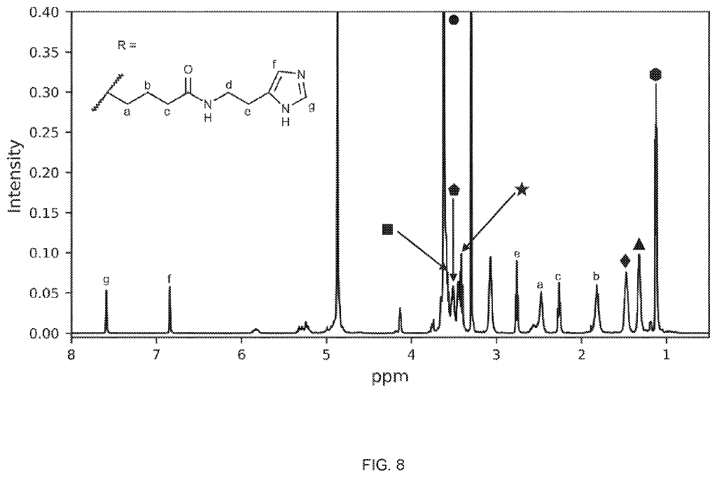

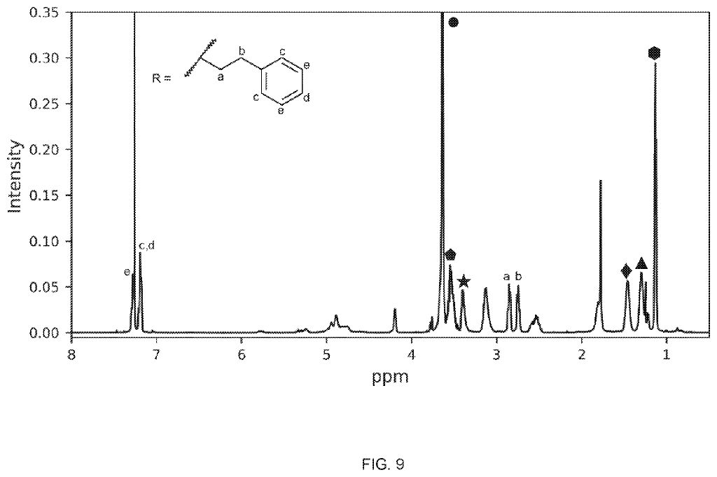

Functionalization of EP(7.7) V with TcFGs was done as described for the functionalization of EP(0.9) V above with the exception that it is performed with varying concentrations due to the limited solubility of the TcFGs. FIGS. 2 to 10 show the NMR spectra of the functionalized EP(7.7) V successfully synthesized. Table 3 shows the concentration of the solvent used for functionalization of EP(7.7) V with various TcFGs.

TABLE 3
Amount of polymer (EP(7.7)V) and corresponding volume and type of
solvent used in the thiol-ene click reaction of the various TcFGs.

| | | |
|---|---|---|
| | Amount of EP(7.7)V | Solvent used for |
| TcFG | functionalized (g) | thiol-ene click reaction |

| | | | |
|---|---|---|---|
| —COOH | 1 | g | Methanol - 10 mL |
| —SO₃Na | 0.5 | g | Methanol - 10 mL |
| —NH₃Cl | 1 | g | Methanol - 10 mL |
| -Cys | 0.5 | g | Methanol - 10 mL |
| -Diol | 0.5 | g | THF - 5 mL |
| -His | 0.5 | g | Methanol - 10 mL |
| -Phenyl | 0.5 | g | THF - 5 mL |

Functionalized thermogelling polymers were further characterized. Table 4 shows the thiol-ene click reaction efficiency and corresponding gel rheological properties of each synthesized functionalized thermogelling polymer.

TABLE 4
Thiol-ene click reaction efficiency and corresponding gel rheological properties. All gels were
prepared at 7 wt % in 1× phosphate-buffered saline. EP(0)V at S/N 1 was used as a control.

| | | Polymer Functionalization and Chemical Composition | | | | | |
|---|---|---|---|---|---|---|---|
| | | | | Initial | | | Functional |
| | Date of | | Reaction | HDDO | Initial vinyl | Click | group |
| | synthesis | Functional | duration | density | density | efficiency | density |
| S/N | (DDMMYY) | Group | (h) | (wt %) | (mmol/g) | (%) | (mmol/g) |
| 1 | [symbol] 2022 | Control | — | 0 | 0 | NA | NA |
| 2 | [symbol] 2022 | -COOH | 3 | 0.38 | 0.067 | 67.70 | 0. [symbol] |
| 3 | [symbol] 2022 | -COOH | 3 | 0.92 | 0.162 | 71.71 | [symbol] .12 |
| 4 | [symbol] 2022 | -COOH | 3 | 4.00 | 0.702 | ~100% (N.D.) | -0.702 |
| 5 | 1 May 2022 | -COOH | 3 | 7.68 | 1.346 | ~100% | -1.34 [symbol] |
| 6 | 20 Apr. 2022 | -SO [symbol] N [symbol] | 3 | 0.92 | 0.162 | [symbol] .42 | [symbol] .14 |
| 7 | 20 Mar. 2022 | -NH [symbol] Cl [symbol] | 3 | 0.92 | 0.162 | 52.25 | 0.085 |
| 8 | 1 May 2022 | -NH [symbol] Cl [symbol] | 3 | 7.68 | 1.346 | [symbol] .42 | 1.11 |
| 9 | 29 Mar. 2022 | -Cys HCl | 3 | 0.92 | 0.162 | [symbol] .27 | 0.00 [symbol] |
| 10 | 31 Mar. 2022 | -Diol | 3 | 0.92 | 0.162 | 28.19 | 0.043 |
| 11 | 31 Mar. 2022 | -Diol | 3 | 7.68 | 1.346 | 92.08 | 1.24 |
| 12 | 31 Mar. 2022 | -Phenyl | 3 | 0.92 | 0.162 | 70.10 | 0.11 |
| 13 | 31 Mar. 2022 | -His | 3 | 0.92 | 0.162 | [symbol] 0.30 | 0.15 |

| | Gel Rheological Properties | | | | | |
|---|---|---|---|---|---|---|
| | | | | | Storage | Complex |
| | Gel | | Gelation | Crossover | modulus | Viscosity |
| | concentration | | temperature | modulus | G′ at 37° C. | at 37° C. |
| S/N | (weight %) | pH | (° C.) | (Pa) | (Pa) | (Pa · s) |
| 1 | 7 | 7.4 | [symbol] | 10.2568 | 1207.3 [symbol] | 195.337 |
| 2 | 7 | 7.51 | 24.43 | 42. [symbol] | [symbol] .074 | 46.3605 |
| 3 | 7 | 7.39 | 2 [symbol] .82 | 42. [symbol] | 209.562 | [symbol] 435 |
| 4 | 7 | 7.45 | 23.18 | 27.4033 | 204.903 | [symbol] |
| 5 | 7 | 7.51 | 29.34 | [symbol] | [symbol] | 11. [symbol] |
| 6 | 7 | 7.41 | 28.63 | 41.3362 | 127.352 | 23.4467 |
| 7 | 7 | 7.40 | 22.38 | 52.8 [symbol] | 402.4 [symbol] | [symbol] 47 |
| 8 | 7 | 7.33 | NA | NA | [symbol] | 1. [symbol] |
| 9 | 7 | 7.3 [symbol] | 23.51 | [symbol] | [symbol] .400 | 57.1131 |
| 10 | 7 | 7.44 | 22. [symbol] | [symbol] | [symbol] | [symbol] .4748 |
| 11 | 7 | 7.43 | 9. [symbol] | 11.33 [symbol] 1 | [symbol] .852 | 113.618 |
| 12 | 7 | 7.48 | [symbol] .43 | 101. [symbol] | 583.865 | 98.32 [symbol] |
| 13 | 7 | 7.33 | 23.83 | [symbol] | 323.743 | [symbol] .9734 |

[symbol]

indicates data missing or illegible when filed

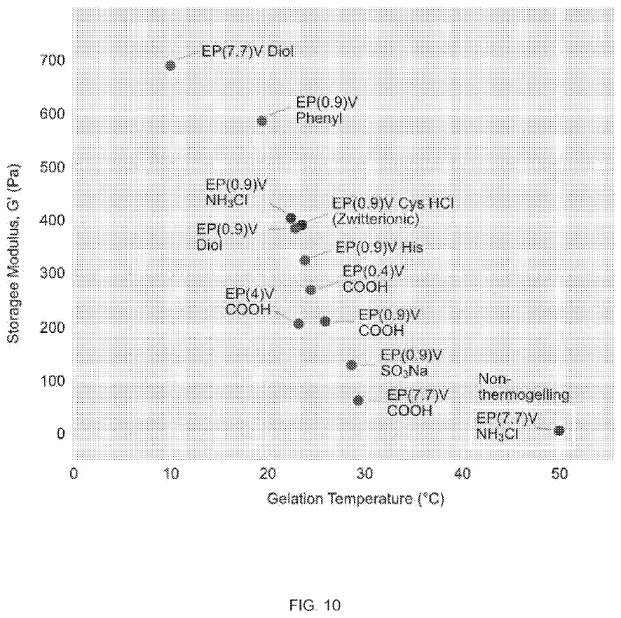

Results show that the efficiency of thiol-ene click reactions varied from 28% to 100%. Nonetheless, quantitative functionalization can be achieved through greater excess of thiols. The results also indicate that the identity of the functional groups appended to the polymer as well as its composition greatly influence its mechanical and gelation properties as shown in FIG. 10. Therefore, being able to tailor its composition is of great importance and a distinctive feature of the present disclosure. In addition to functional and composition versatility, various molecular weights could be achieved by adjusting the polymerization time, and poly(caprolactone) could be copolymerized to provide biodegradability in expanding the adaptability of this synthetic strategy for developing multifunctional thermogels.

### Example 3: Synthesis of His-SH TcFG

The general steps for functionalizing a thermogelling polymer in accordance with various embodiments disclosed herein may further include synthesizing the compound comprising one or more TcFGs (for the example below, His-SH was synthesized) prior to the functionalization step.

An example of synthesizing the compound comprising one or more TcFGs in accordance with various embodiments disclosed herein is described in detail as follows.

Firstly, the synthesis of His-SH commenced with weighing 500 mg of histamine into a 50 mL round bottom flask. Subsequently, 7.5 mL of anhydrous acetonitrile and 505.4 mg of γ-thiobutyrolactone were introduced to the flask. The reaction was then allowed to proceed at a temperature of 95° C. for a duration of 16 hours. Following the completion of the reaction, precipitates formed were filtered and subjected to three washes, each with 10 mL of acetonitrile. The resulting product was then dried under vacuum conditions at 60° C. for a period of 16 hours to obtain His-SH.

### Discussions of Examples

In Example 1, a highly streamlined yet versatile strategy for producing a library of functional polyurethane-based thermo-responsive polymers using polyaddition and thiol-ene click chemistry is shown. To synthesize the vinyl-containing thermogels, poly(ethylene glycol) (PEG, Mn ~2050), poly(propylene glycol) (PPG, Mn ~2000), and 1,5-hexadiene-3,4-diol (HDDO) were polymerized in the presence hexamethylene diisocyanate (HMDI) (with molar ratio of diol:HMDI=1:1.01) at 110° C. in anhydrous toluene and catalytic amounts of dibutyltin dilaurate (DBTL). The weight percentage of vinyl groups incorporated into the polymer can be tailored by controlling the initial amount of HDDO in the reaction. The resulting vinyl-containing polymers were then reacted with the desired TcFG in the presence of 2-hydroxy-4′-(2-hydroxyethoxy)-2-methylpropiophenone) and ultraviolet light (365 nm). By introducing these vinyl groups along the chain, it is possible to readily attach a broad range of functional groups at a high density compared to existing strategies such as chain end modifications.

Scheme 3 below provides an illustration of EPV indicating a PEG and PPG block copolymer containing reactive vinyl handles derived from integrating HDDO into the polymer backbone. EPV can then be subsequently functionalized by thiol-containing functional groups via thiol-ene click reaction. A non-exhaustive list of functional groups is demonstrated in this disclosure.

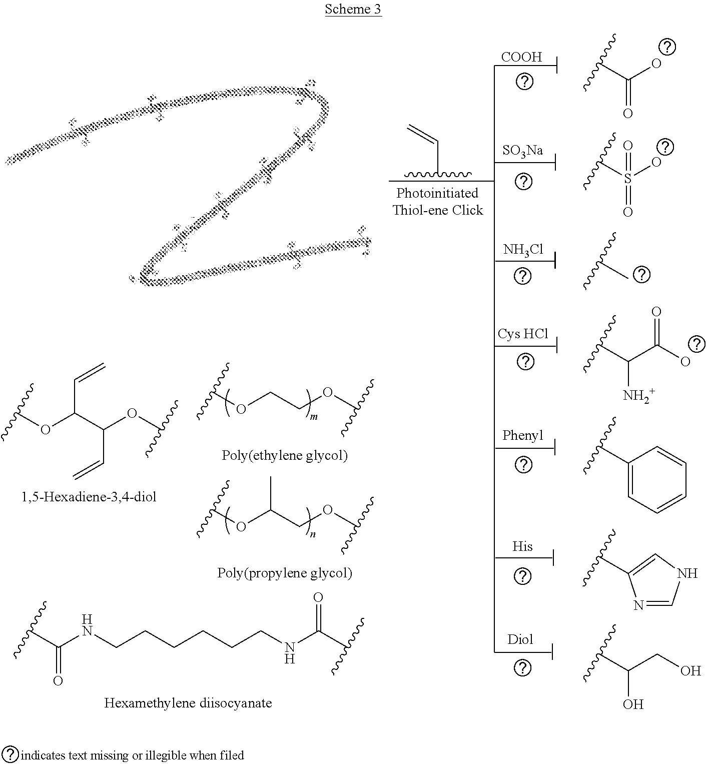

The Examples have showcased the versatility of this strategy with the functionalization of thioglycolic acid (anionic) and sodium 2-mercaptoethanesulfonate (anionic), an acid, and an acid salt with pKa's of ~5 and ~-7. The latter would therefore be pH-insensitive and remain negatively charged across the range of physiological pH. The functionalizations of 2-aminoethanethiol hydrochloride (cationic), cysteine hydrochloride (zwitterionic), 2-phenylethanethiol (aromatic), His-SH (imidazole, metal-binding), and 10 thioglycerol (dihydroxy, hydrogen-bonding) have also been demonstrated.

In summary, the Examples detail the streamlined two-stage synthesis process that allows convenient and efficient production of thermogelling multi-block copolymers with a multitude of functional groups and control over their composition. The versatility of this method is showcased in the Examples by the synthesis of thermogels containing carboxylic acids, sulphonates, amines, cysteines (zwitterionic), phenyl rings, diols, and histamines (imidazole). Utilizing embodiments of the disclosed method, it is envisaged that multifunctional thermogels can also be achieved by mixing-and-matching thiol-containing moieties, e.g. carboxylic acid for sustained release of cationic drugs and histamine for metal-coordinated crosslinking, creating a truly versatile platform for thermogels with unique and multiple functionalities.

### Applications

Embodiments of the present disclosure provide a versatile synthetic platform for multifunctional polyurethane thermogels via thiol-ene click chemistry. Advantageously, embodiments of the present disclosure provide a novel functionalizable polymer backbone design for thermogels.

Embodiments of the present disclosure also provide a method for post-polymerization functionalization of EPV with thiol-ene click chemistry. As a result, a library of thermogels with an unprecedented variety of functionalities and functional groups including charged, pH-responsive, zwitterionic, aromatic rings capable of cation-IT interactions, hydrogen-bonding, and metal-binding groups may be obtained in a facile manner.

Advantageously, the number of functional groups in embodiments of the disclosed thermogels may be varied. For example, this has been demonstrated in the synthesis of varying compositions of vinyl-containing thermogelling polyurethanes made up of PEG, PPG and Vinyl-containing 1,5-hexadiene-3,4-diol (EPV). These thermogels can be modified post-polymerization (e.g., EP(0) V, EP(0.4) V, EP(0.9) V, EP(4) V, EP(7.7) V) which in turn results in varying the number of functional groups on each polymer chain (e.g., EP(0.4) V—COOH, EP(0.9) V—COOH, EP(4) V—COOH, EP(7.7) V—COOH). Therefore, embodiments of the methods disclosed herein also provide for the synthesis and purification of EPV as a versatile backbone with tunable functional group type and density for multifunctional thermogelling polymers.

Advantageously, embodiments of the presently disclosed method provide a synthetic platform for preparing thermogelling polyurethanes that facilitates easy post-polymerization functionalization of these polymers with a library of functional groups, thereby imparting a range of functional properties. This easy and versatile modification of the thermogel, allows for tunability of functional group density, and offers an inert thermogelling backbone after functionalization. These easily-tuned gel properties and applications include pH-responsiveness, mechanical resilience, bioadhesion, etc. Such tuning may be achieved by simply changing the type and ratio of functionalization. In various embodiments, this is first enabled through the introduction of vinyl-containing diol monomers that would provide the reactive handle for subsequent functionalization, into the amphiphilic thermogelling backbone. This allows the easy functionalization of a wide range of thiol-containing functional groups using thiol-ene click chemistry. Thereafter, convenient tailoring of functional group density may be achieved by varying vinyl-content and degree of functionalization. In other words, the unique approach of integrating vinyl-containing monomers into the main chain of polyurethane thermogels may be subsequently leveraged for post-synthesis functionalization. This can lead to thermogels with a broadened range of properties when compared with traditional PEG-based thermogels, allowing for customizable multifunctionality and temperature-responsiveness for an expanded range of applications normally unsuitable for PEG-based thermogels. Examples of successfully incorporated functional groups and their applications (non-exhaustive list) include:
Anionic carboxylates and sulfonates for pH-dependent and pH-independent sustained drug release;
Cationic amines for pH-dependent sustained drug release;
Zwitterionic (cysteine) for pH-dependent sustained drug release and antifouling applications;
Phenyl rings for pi-cation interactions and dual crosslinked gels;
Imidazole (histamine) for metal binding and dual crosslinked gels; and
Diols for hydrogen-bonding and dual crosslinked gels.

Advantageously, the resulting water solubility at lower temperatures and thermogelling behaviour at higher temperatures of the library of functionalized polymers obtained from embodiments of the methods disclosed herein add another functional dimensionality to the thermo-responsive gel and cannot be easily predicted a priori. For example, the highly charged cationic and anionic polymers obtained through embodiments of the method disclosed herein retain the capability to form gels at 37° C., while the hydrophobic aromatic phenyl group increases the storage modulus of the thermogels at 37° C. while remaining soluble at low temperatures. The vast array of novel and unexpected thermogel properties accessible using embodiments of the methods disclosed herein has the potential for exploitation in many lucrative new biomedical applications traditionally unsuitable for thermogels.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A thermogelling polymer comprising one or more repeating units derived from a hydrophilic polymer, one or more repeating units derived from a thermoresponsive polymer, and one or more repeating units derived from a vinyl-containing diol monomer,
wherein the one or more repeating units derived from a hydrophilic polymer, the one or more repeating units derived from a thermoresponsive polymer, and the one or more repeating units derived from a vinyl-containing diol monomer are chemically coupled together by at least one of urethane/carbamate, carbonate, ester linkages or combinations thereof,
optionally wherein the thermogelling polymer has a polydensity index (PDI) falling in a range of from 1 to 2.

2. The thermogelling polymer according to claim 1, wherein
the one or more repeating units derived from a hydrophilic polymer is/are represented by general formula (1); the one or more repeating units derived from a thermoresponsive polymer is/are represented by general formula (2); the one or more repeating units derived from a vinyl-containing diol monomer is/are represented by general formula (3); and the at least one of urethane/carbamate, carbonate, ester linkages or combinations thereof is/are represented by general formula (5): 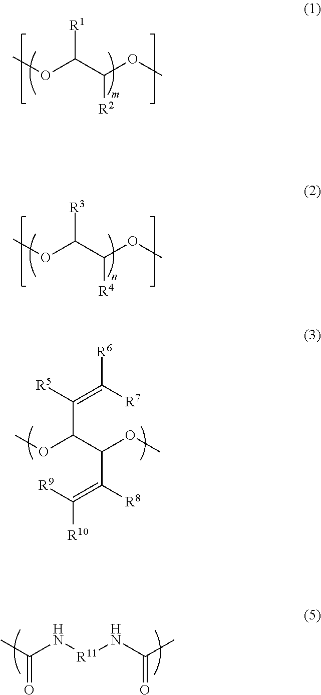
wherein
R¹ to R¹⁰ are each independently selected from H, alkyl, or optionally substituted alkyl;
$$m \geq 1 ;$$
$$n \geq 1 ;$$
R¹¹ is alkyl or optionally substituted alkyl.

3. The thermogelling polymer according to claim 2, wherein general formula (1) comprises poly(ethylene glycol) (PEG), general formula (2) comprises poly(propylene glycol) (PPG), general formula (3) comprises 1,5-hexadiene-3,4-diol (HDDO), and general formula (5) comprises hexamethylene diisocyanate (HMDI).

4. The thermogelling polymer according to claim 2, wherein general formula (1) is present in an amount of between 0 mass % to 90 mass % of the thermogelling polymer, general formula (2) is present in an amount of between 0 mass % to 90 mass % of the thermogelling polymer, general formula (3) is present in an amount of from 0.1 mass % to 40% of the thermogelling polymer, and general formula (5) is present in an amount of from 10 mass % to 60 mass % of the thermogelling polymer; and/or the thermogelling polymer comprises 0.01 mmol/g to 7 mmol/g of vinyl groups (e.g., —CH═CH₂).

5. (canceled)

6. (canceled)

7. The thermogelling polymer according to claim 1, wherein one or more vinyl groups of the thermogelling polymer have been further functionalized.

8. A functionalized thermogelling polymer comprising one or more repeating units derived from a hydrophilic polymer, one or more repeating units derived from a thermoresponsive polymer, and one or more repeating units derived from a functionalized vinyl-containing diol monomer,
wherein the one or more repeating units derived from a hydrophilic polymer, the one or more repeating units derived from a thermoresponsive polymer, and the one or more repeating units derived from a functionalized vinyl-containing diol monomer are chemically coupled together by at least one of urethane/carbamate, carbonate, ester linkages or combinations thereof,
optionally wherein the functionalized thermogelling polymer has one or more of the following properties: a pH value falling in a range of from 1 to 10; a gelation temperature falling in a range of from 4° C. to 60° C.; a crossover modulus falling in a range of from 5 Pa to 1000 Pa; a storage modulus (G′) in a range of from 4 Pa to 5000 Pa at about 37° C.; or a complex viscosity falling in a range of from 1 Pa·s to 500 Pa·s at about 37° C.

9. The functionalized thermogelling polymer according to claim 8, wherein
the one or more repeating units derived from a hydrophilic polymer is/are represented by general formula (1); the one or more repeating units derived from a thermoresponsive polymer is/are represented by general formula (2); the one or more repeating units derived from a functionalized vinyl-containing diol monomer is/are represented by general formula (4); and the at least one of urethane/carbamate, carbonate, ester linkages or combinations thereof is/are represented by general formula (5): 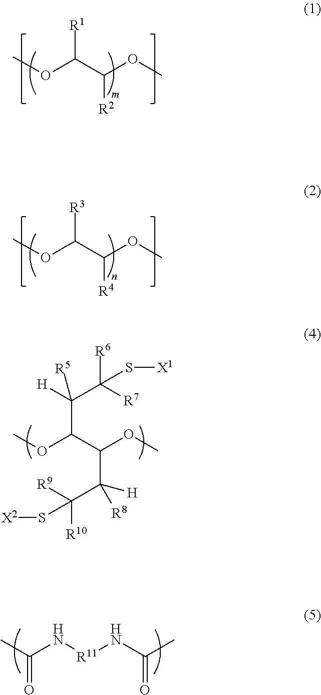
wherein
R¹ to R¹⁰, m and n are as defined above;
X¹—S and X²—S each independently comprises a thiol-containing functional group; R¹¹ is alkyl or optionally substituted alkyl.

10. The functionalized thermogelling polymer according to claim 9, wherein general formula (1) comprises poly(ethylene glycol) (PEG), general formula (2) comprises poly(propylene glycol) (PPG), general formula (3) comprises 1,5-hexadiene-3,4-diol (HDDO), and general formula (5) comprises hexamethylene diisocyanate (HMDI).

11. The functionalized thermogelling polymer according to claim 9, wherein X¹ and X² are each independently a moiety comprising one or more of a carboxylic acid, an amino acid, a sulfonate, an alkylsulfonate, an aminehydrochloride, an alcohol, a diol, a benzene, an alkylbenzene, or derivatives thereof,
optionally wherein X¹ and X² are each independently selected from the group consisting of the following structures or their derivatives thereof:

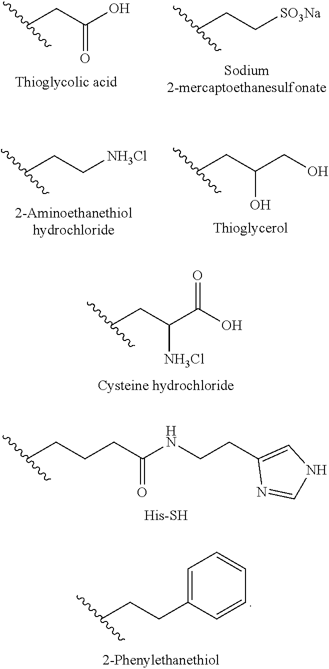

12. (canceled)

13. (canceled)

14. The functionalized thermogelling polymer of claim 9, wherein the functionalized thermogelling polymer has a functional group density for X¹ and/or X² falling in a range of from 0.01 mmol/g to 7 mmol/g.

15. A method of preparing a thermogelling polymer of claim 1, the method comprising:
coupling one or more hydrophilic polymers, one or more thermoresponsive polymers, and one or more vinyl-containing diol monomers in the presence of a coupling agent to obtain a thermogelling polymer,
wherein the coupling agent comprises a urethane/carbamate forming agent, carbonate forming agent, ester forming agent, or combinations thereof,
optionally wherein the coupling agent is a diisocyanate selected from the group consisting of hexamethylene diisocyanate (HMDI), tetramethylene diisocyanate, cyclohexane diisocyanate, tetramethylxylene diisocyanate, dodecylene diisocyanate, tolylene 2,4-diisocyanate, and tolylene 2,6-diisocyanate,
optionally wherein the coupling step is performed at an elevated temperature falling in a range of from 60° C. to 150° C.; and/or the coupling step is carried out for at least 2 hours; and/or the coupling step is carried out in the presence of an anhydrous solvent selected from the group consisting of toluene, benzene, and xylene.

16. The method according to claim 15, wherein
the one or more hydrophilic polymers is/are represented by general formula (6); the one or more thermoresponsive polymers is/are represented by general formula (7); and the one or more vinyl-containing diol monomers is/are represented by general formula (8), 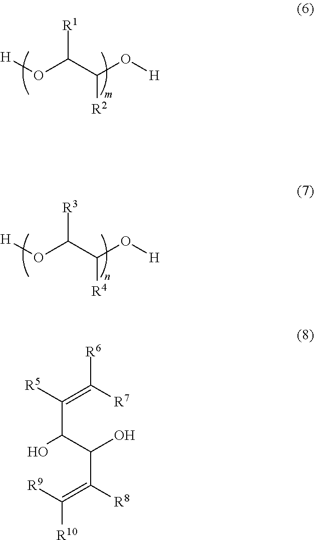
wherein R¹ to R¹⁰, m and n are as defined above.

17. The method according to claim 16, wherein general formula (6) comprises poly(ethylene glycol) (PEG), general formula (7) comprises poly(propylene glycol) (PPG), and general formula (8) comprises 1,5-hexadiene-3,4-diol (HDDO).

18. The method according to claim 16, wherein the one or more hydrophilic polymers represented by general formula (6), one or more thermoresponsive polymers represented by general formula (7), and one or more vinyl-containing diol monomers represented by general formula (8) are mixed in a mass ratio of 1-20:1-10:0.01-3.

19. (canceled)

20. (canceled)

21. (canceled)

22. The method according to claim 15, wherein the coupling agent is added in a ratio of 1-2:1-2 relative to one or more vinyl-containing diol monomers represented by general formula (8).

23. (canceled)

24. The method according to claim 15, wherein the coupling step is carried out in the presence of a metal-containing or non-metal containing catalyst that is capable of catalysing formation of urethane/carbamate, carbonate, ester linkages, or combinations thereof from alcohols and a suitable isocyanate precursor or derivatives thereof,
optionally wherein the metal-containing catalyst comprises a tin-based catalyst and/or a zinc-based catalyst.

25. (canceled)

26. The method according to claim 15, wherein the method further comprises functionalizing the thermogelling polymer with one or more thiols to obtain a functionalized thermogelling polymer with thiol-containing functional groups X¹—S and X²—S, where X¹ and X² each independently comprises a functional group,
optionally wherein X¹ and X² are each independently a moiety comprising one or more of a carboxylic acid, an amino acid, a sulfonate, an alkylsulfonate, an aminehydrochloride, an alcohol, a diol, a benzene, an alkylbenzene, or derivatives thereof,
and optionally wherein X¹ and X² are each independently selected from the group consisting of the following structures or their derivatives thereof:

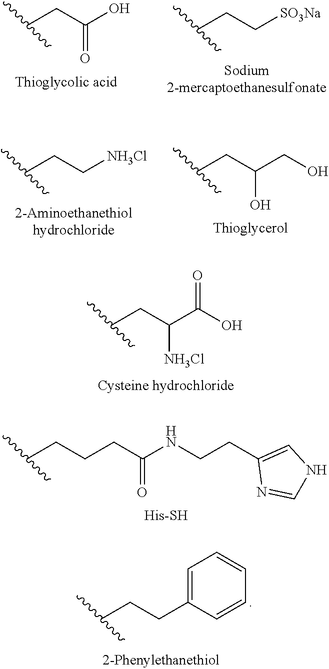

27. (canceled)

28. (canceled)

29. The method according to claim 26, wherein functionalizing the thermogelling polymer is carried out in the presence of a radical initiator, photoinitiator, and/or ultraviolet (UV) light, optionally wherein functionalizing the thermogelling polymer is carried out in the presence of UV light for at least 30 minutes;
and/or functionalizing the thermogelling polymer is carried out in the presence of an anhydrous solvent, optionally wherein the anhydrous solvent is selected from the group of anhydrous tetrahydrofuran (THF) and anhydrous methanol.

30. (canceled)

31. (canceled)

32. The method according to claim 26, wherein the one or more thiols is added in a molar ratio of 20-1:1-20, optionally 3:1, relative to the vinyl groups in the thermogelling polymer,
and/or the photoinitiator is added in a molar ratio of at least 3-1:1-3, optionally 2:1 relative to the vinyl groups in the functionalized thermogelling polymer, and optionally wherein the photoinitiator comprises 2-hydroxy-4′-(2-hydroxyethoxy)-2-methylpropiophenone.

33. (canceled)

34. (canceled)

35. The method according to claim 26, wherein functionalizing the thermogelling polymer with one or more thiols has a functionalization efficiency falling in a range of from 5% to 100%.
